SEQUENCE LISTING (29 sequences)

SEQ ID NO: 1 - protein, 213 aa, Artificial Sequence
  Synthetic Polypeptide
DIQMTQSPSSLSASVGDRVTITCSASQDISNYLNWYQQKPGKAPKVLIYFTSSLHSGVPSRFSGSGSGTDFTLTISSLQPEDFATYYCQQYSTVPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGE

SEQ ID NO: 2 - protein, 224 aa, Artificial Sequence
  Synthetic Polypeptide
EVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPK

SEQ ID NO: 3 - protein, 229 aa, Artificial Sequence
  Synthetic Polypeptide
SCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 4 - protein, 224 aa, Artificial Sequence
  Synthetic Polypeptide
EVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTNVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPK

SEQ ID NO: 5 - protein, 213 aa, Artificial Sequence
  Synthetic Polypeptide
DIQMTQSPSSLSASVGDRVTITCSASQDISNYLNWYQQKPGKAPKVLIYFTSSLHSGVPSRFSGSGSGTDFTLTISSLQPEDFATYYCQQYSTVPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACNVTHQGLSSPVTKSFNRGE

SEQ ID NO: 6 - protein, 213 aa, Artificial Sequence
  Synthetic Polypeptide
DIQMTQSPSSLSASVGDRVTITCSASQDISNYLNWYQQKPGKAPKVLIYFTSSLHSGVPSRFSGSGSGTDFTLTISSLQPEDFATYYCQQYSTVPWTFGQGTKVEIKRTVAAPSVFIFPPSDEQLKSGTASVVCLLNNFYPREAKVQWKVDNALQSGNSNESVTEQDSKDSTYSLSSTLTLSKADYEKHKVYACEVTHQGLSSPVTKSFNRGE

SEQ ID NO: 7 - protein, 213 aa, Artificial Sequence
  Synthetic Polypeptide
DIQMTQSPSSLSASVGDRVTITCSASQDISNYLNWYQQKPGKAPKVLIYFTSSLHSGVPSRFSGSGSGTDFTLTISSLQPEDFATYYCQQYSTVPWTFGQGTKVEIKRTVAAPSVFIFPPSDEQLKSGTASVVCLLNNFYPREAKVQWKVDNALQSGNSSESVTEQDSKDSTYSLSSTLTLSKADYEKHKVYACEVTHQGLSSPVTKSFNRGE

SEQ ID NO: 8 - protein, 232 aa, Homo sapiens
MNFLLSWVHWSLALLLYLHHAKWSQAAPMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQEKKSVRGKGKGQKRKRKKSRYKSWSVYVGARCCLMPWSLPGPHPCGPCSERRKHLFVQDPQTCKCSCKNTDSRCKARQLELNERTCRCDKPRR

SEQ ID NO: 9 - protein, 224 aa, Artificial Sequence
  Synthetic Polypeptide
EVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTXVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPK

SEQ ID NO: 10 - protein, 213 aa, Artificial Sequence
  Synthetic Polypeptide
DIQMTQSPSSLSASVGDRVTITCSASQDISNYLNWYQQKPGKAPKVLIYFTSSLHSGVPSRFSGSGSGTDFTLTISSLQPEDFATYYCQQYSTVPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACXVTHQGLSSPVTKSFNRGE

SEQ ID NO: 11 - protein, 213 aa, Artificial Sequence
  Synthetic Polypeptide
DIQMTQSPSSLSASVGDRVTITCSASQDISNYLNWYQQKPGKAPKVLIYFTSSLHSGVPSRFSGSGSGTDFTLTISSLQPEDFATYYCQQYSTVPWTFGQGTKVEIKRTVAAPSVFIFPPSDEQLKSGTASVVCLLNNFYPREAKVQWKVDNALQSGNSXESVTEQDSKDSTYSLSSTLTLSKADYEKHKVYACEVTHQGLSSPVTKSFNRGE

SEQ ID NO: 12 - protein, 213 aa, Artificial Sequence
  Synthetic Polypeptide
DIQMTQSPSSLSASVGDRVTITCSASQDISNYLNWYQQKPGKAPKVLIYFTSSLHSGVPSRFSGSGSGTDFTLTISSLQPEDFATYYCQQYSTVPWTFGQGTKVEINRTVAAPSVFIFPPSDEQLKSGTASVVCLLNNFYPREAKVQWKVDNALQSGNSSESVTEQDSKDSTYSLSSTLTLSKADYEKHKVYACEVTHQGLSSPVTKSFNRGE

SEQ ID NO: 13 - protein, 213 aa, Artificial Sequence
  Synthetic Polypeptide
DIQMTQSPSSLSASVGDRVTITCSASQDISNYLNWYQQKPGKAPKVLIYFTSSLHSGVPSRFSGSGSGTDFTLTISSLQPEDFATYYCQQYSTVPWTFGQGTKVEIKRTVAAPSVFIFPPSDEQLKSGTASVVCLLNNFSPREAKVQWKVDNALQSGNSSESVTEQDSKDSTYSLSSTLTLSKADYEKHKVYACEVTHQGLSSPVTKSFNRGE

SEQ ID NO: 14 - protein, 213 aa, Artificial Sequence
  Synthetic Polypeptide
DIQMTQSPSSLSASVGDRVTITCSASQDISNYLNWYQQKPGKAPKVLIYFTSSLHSGVPSRFSGSGSGTDFTLTISSLQPEDFATYYCQQYSTVPWTFGQGTKVEIKRTVAAPSVFIFPPSDEQLKSGTASVVCLLNNFYPREAKVQWKVDNALQSGNSSESVTEQDSNDSTYSLSSTLTLSKADYEKHKVYACEVTHQGLSSPVTKSFNRGE

SEQ ID NO: 15 - protein, 213 aa, Artificial Sequence
  Synthetic Polypeptide
DIQMTQSPSSLSASVGDRVTITCSASQDISNYLNWYQQKPGKAPKVLIYFTSSLHSGVPSRFSGSGSGTDFTLTISSLQPEDFATYYCQQYSTVPWTFGQGTKVEIKRTVAAPSVFIFPPSDEQLKSGTASVVCLLNNFYPREAKVQWKVDNALQSGNSSESVTEQDSKNSTYSLSSTLTLSKADYEKHKVYACEVTHQGLSSPVTKSFNRGE

SEQ ID NO: 16 - protein, 213 aa, Artificial Sequence
  Synthetic Polypeptide
DIQMTQSPSSLSASVGDRVTITCSASQDISNYLNWYQQKPGKAPKVLIYFTSSLHSGVPSRFSGSGSGTDFTLTISSLQPEDFATYYCQQYSTVPWTFGQGTKVEIXRTVAAPSVFIFPPSDEQLKSGTASVVCLLNNFYPREAKVQWKVDNALQSGNSSESVTEQDSKDSTYSLSSTLTLSKADYEKHKVYACEVTHQGLSSPVTKSFNRGE

SEQ ID NO: 17 - protein, 213 aa, Artificial Sequence
  Synthetic Polypeptide
DIQMTQSPSSLSASVGDRVTITCSASQDISNYLNWYQQKPGKAPKVLIYFTSSLHSGVPSRFSGSGSGTDFTLTISSLQPEDFATYYCQQYSTVPWTFGQGTKVEIKRTVAAPSVFIFPPSDEQLKSGTASVVCLLNNFXPREAKVQWKVDNALQSGNSSESVTEQDSKDSTYSLSSTLTLSKADYEKHKVYACEVTHQGLSSPVTKSFNRGE

SEQ ID NO: 18 - protein, 213 aa, Artificial Sequence
  Synthetic Polypeptide
DIQMTQSPSSLSASVGDRVTITCSASQDISNYLNWYQQKPGKAPKVLIYFTSSLHSGVPSRFSGSGSGTDFTLTISSLQPEDFATYYCQQYSTVPWTFGQGTKVEIKRTVAAPSVFIFPPSDEQLKSGTASVVCLLNNFYPREAKVQWKVDNALQSGNSSESVTEQDSXDSTYSLSSTLTLSKADYEKHKVYACEVTHQGLSSPVTKSFNRGE

SEQ ID NO: 19 - protein, 213 aa, Artificial Sequence
  Synthetic Polypeptide
DIQMTQSPSSLSASVGDRVTITCSASQDISNYLNWYQQKPGKAPKVLIYFTSSLHSGVPSRFSGSGSGTDFTLTISSLQPEDFATYYCQQYSTVPWTFGQGTKVEIKRTVAAPSVFIFPPSDEQLKSGTASVVCLLNNFYPREAKVQWKVDNALQSGNSSESVTEQDSKXSTYSLSSTLTLSKADYEKHKVYACEVTHQGLSSPVTKSFNRGE

SEQ ID NO: 20 - protein, 214 aa, Artificial Sequence
  Synthetic Polypeptide
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 21 - protein, 220 aa, Artificial Sequence
  Synthetic Polypeptide
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEP

SEQ ID NO: 22 - protein, 231 aa, Artificial Sequence
  Synthetic Polypeptide
PKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 23 - protein, 220 aa, Artificial Sequence
  Synthetic Polypeptide
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTNVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEP

SEQ ID NO: 24 - protein, 214 aa, Artificial Sequence
  Synthetic Polypeptide
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSNESVTEQDSKDSTYSLSSTLTLSKADYEKHKVYACEVTHQGLSSPVTKSFNRGEC

SEQ ID NO: 25 - protein, 214 aa, Artificial Sequence
  Synthetic Polypeptide
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACNVTHQGLSSPVTKSFNRGEC

SEQ ID NO: 26 - protein, 1255 aa, Homo sapiens [sequence omitted]

SEQ ID NO: 27 - protein, 220 aa, Artificial Sequence
  Synthetic Polypeptide
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTXVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEP

SEQ ID NO: 28 - protein, 214 aa, Artificial Sequence
  Synthetic Polypeptide
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSXESVTEQDSKDSTYSLSSTLTLSKADYEKHKVYACEVTHQGLSSPVTKSFNRGEC

SEQ ID NO: 29 - protein, 214 aa, Artificial Sequence
  Synthetic Polypeptide
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACXVTHQGLSSPVTKSFNRGEC

(19) United States
(12) Patent Application Publication
(10) Pub. No.: US 2017/0114127 A1
(43) Pub. Date: Apr. 27, 2017

(54) VEGF-A-BINDING PROTEINS AND HER2-BINDING PROTEINS WITH ENHANCED STABILITY AGAINST AGGREGATION

(71) Applicant: Massachusetts Institute of Technology, Cambridge, MA (US)

(72) Inventors: Bernhardt Levy Trout, Lexington, MA (US); Neeraj Jagdish Agrawal, Thousand Oaks, CA (US); Fabienne Courtois, Boston, MA (US)

(73) Assignee: Massachusetts Institute of Technology, Cambridge, MA (US)

(21) Appl. No.: 15/299,861

(22) Filed: Oct. 21, 2016

Related U.S. Application Data
(60) Provisional application No. 62/247,274, filed on Oct. 28, 2015.
(60) Provisional application No. 62/245,169, filed on Oct. 22, 2015.

(51) Int. Cl. C07K 16/22; C07K 16/28

(52) U.S. Cl. CPC C07K 16/22; C07K 16/2863; C07K 2317/24; C07K 2317/41; C07K 2317/94; C07K 2317/92; C07K 2317/55

(57) ABSTRACT

The present disclosure provides VEGF-A-binding proteins and HER-2-binding proteins having a reduced tendency to aggregate. Compositions and methods of use are also provided.

### RELATED APPLICATIONS

This application claims the benefit under 35 U.S.C. §119(e) of U.S. provisional application No. 62/247,274, filed Oct. 28, 2015 and U.S. provisional application No. 62/245,169, filed Oct. 22, 2015, each of which is incorporated by reference herein in its entirety.

### BACKGROUND

Subcutaneous injection of therapeutic proteins requires a high protein concentration in the final solution to be injected. Achieving the high protein concentration necessary for subcutaneous delivery can be problematic due to protein aggregation. Aggregation is the result of intermolecular interactions and, thus, is enhanced by high protein concentrations. The presence of protein aggregates in an injected solution, even in small doses, poses a threat of an immune response that can reduce the efficacy of the protein over time and, more importantly, has the potential to elicit adverse reactions.

### SUMMARY

The present disclosure provides proteins that are less prone to aggregation as compared to existing proteins. Results presented herein show that specific mutations at particular amino acids in Vascular Endothelial Growth Factor A (VEGF-A)-binding proteins reduce the tendency of the proteins to aggregate in solution. The present disclosure also provides Human Epidermal Growth Factor Receptor 2 (HER2)-binding proteins having specific mutations at particular amino acids for reducing the tendency of the proteins to aggregate in solution.

Some embodiments provide VEGF-A-binding protein comprising a heavy chain domain having an amino acid sequence of SEQ ID NO: 2, and a light chain domain having an amino acid sequence of SEQ ID NO: 5, SEQ ID NO: 6, SEQ ID NO: 7, SEQ ID NO: 12, SEQ ID NO: 13, SEQ ID NO: 14 or SEQ ID NO: 15. In some embodiments, the light chain domain has an amino acid sequence of SEQ ID NO: 5. In some embodiments, the light chain domain has an amino acid sequence of SEQ ID NO: 6. In some embodiments, the light chain domain has an amino acid sequence of SEQ ID NO: 7. In some embodiments, the light chain domain has an amino acid sequence of SEQ ID NO: 12. In some embodiments, the light chain domain has an amino acid sequence of SEQ ID NO: 13. In some embodiments, the light chain domain has an amino acid sequence of SEQ ID NO: 14. In some embodiments, the light chain domain has an amino acid sequence of SEQ ID NO: 15.

Some embodiments provide a VEGF-A-binding protein comprising a heavy chain domain having an amino acid sequence of SEQ ID NO: 4, and a light chain domain having an amino acid sequence of SEQ ID NO: 1.

Some embodiments provide a VEGF-A-binding protein comprising a heavy chain domain having an amino acid sequence of SEQ ID NO: 4, and a light chain domain having an amino acid sequence of SEQ ID NO: 5, SEQ ID NO: 6, SEQ ID NO:7, SEQ ID NO: 12, SEQ ID NO: 13, SEQ ID NO: 14 or SEQ ID NO: 15. In some embodiments, the light chain domain has an amino acid sequence of SEQ ID NO: 5. In some embodiments, the light chain domain has an amino acid sequence of SEQ ID NO: 6. In some embodiments, the light chain domain has an amino acid sequence of SEQ ID NO: 7. In some embodiments, the light chain domain has an amino acid sequence of SEQ ID NO: 12. In some embodiments, the light chain domain has an amino acid sequence of SEQ ID NO: 13. In some embodiments, the light chain domain has an amino acid sequence of SEQ ID NO: 14. In some embodiments, the light chain domain has an amino acid sequence of SEQ ID NO: 15.

Some embodiments provide a VEGF-A-binding protein comprising a heavy chain domain having an amino acid sequence of SEQ ID NO: 4. Some embodiments provide a VEGF-A-binding protein comprising a light chain domain having an amino acid sequence of SEQ ID NO: 5. Some embodiments provide a VEGF-A-binding protein comprising a light chain domain having an amino acid sequence of SEQ ID NO: 6. Some embodiments provide a VEGF-A-binding protein comprising a light chain domain having an amino acid sequence of SEQ ID NO: 7. Some embodiments provide a VEGF-A-binding protein comprising a light chain domain having an amino acid sequence of SEQ ID NO: 12. Some embodiments provide a VEGF-A-binding protein comprising a light chain domain having an amino acid sequence of SEQ ID NO: 13. Some embodiments provide a VEGF-A-binding protein comprising a light chain domain having an amino acid sequence of SEQ ID NO: 14. Some embodiments provide a VEGF-A-binding protein comprising a light chain domain having an amino acid sequence of SEQ ID NO: 15.

In some embodiments, the VEGF-A-binding protein comprises an Fc domain having an amino acid sequence of SEQ ID NO: 3.

Some embodiments provide a HER2-binding protein comprising a heavy chain domain having an amino acid sequence of SEQ ID NO: 21, and a light chain domain having an amino acid sequence of SEQ ID NO: 24 or SEQ ID NO: 25. In some embodiments, the light chain domain has an amino acid sequence of SEQ ID NO: 24. In some embodiments, the light chain domain has an amino acid sequence of SEQ ID NO: 25.

Some embodiments provide a HER2-binding protein comprising a heavy chain domain having an amino acid sequence of SEQ ID NO: 23, and a light chain domain having an amino acid sequence of SEQ ID NO: 20.

Some embodiments provide a HER2-binding protein comprising a heavy chain domain having an amino acid sequence of SEQ ID NO: 23, and a light chain domain having an amino acid sequence of SEQ ID NO: 24 or SEQ ID NO: 25. In some embodiments, the light chain domain has an amino acid sequence of SEQ ID NO: 24. In some embodiments, the light chain domain has an amino acid sequence of SEQ ID NO: 25.

Some embodiments provide a HER2-binding protein comprising a heavy chain domain having an amino acid sequence of SEQ ID NO: 23. Some embodiments provide a HER2-binding protein comprising a light chain domain having an amino acid sequence of SEQ ID NO: 24. Some embodiments provide a HER2-binding protein comprising a light chain domain having an amino acid sequence of SEQ ID NO: 25.

In some embodiments. the HER2-binding protein comprises an Fc domain having an amino acid sequence of SEQ ID NO: 22.

In some embodiments, the VEGF-A-binding protein or HER2-binding protein is in the form of a monoclonal antibody. The monoclonal antibody may be, for example, a chimeric monoclonal antibody, human monoclonal antibody or humanized monoclonal antibody.

In some embodiments, the VEGF-A-binding protein or HER2-binding protein is in the form of a fusion protein.

In some embodiments, the VEGF-A-binding protein or HER2-binding protein is in the form of a the protein is in the form of a Fab antibody fragment, single-chain Fv antibody fragment, or minibody.

In some embodiments, the VEGF-A-binding protein or HER2-binding protein is conjugated to a diagnostic agent or a therapeutic agent.

In some embodiments, the VEGF-A-binding protein or HER2-binding protein is glycosylated or hyperglycosylated.

Some embodiments provide a composition comprising a VEGF-A-binding protein of the present disclosure. Some embodiments provide a composition comprising a HER2-binding protein of the present disclosure.

In some embodiments, the VEGF-A-binding protein or HER2-binding protein is present at a concentration of 50 mg/ml to 250 mg/ml. In some embodiments, the protein is present at a concentration of 100 mg/ml to 200 mg/ml. In some embodiments, the protein is present at a concentration of 110 mg/ml to 150 mg/ml. In some embodiments, the protein is present at a concentration of 120 mg/ml. In some embodiments, the protein is present at a concentration of 130 mg/ml. In some embodiments, the protein is present at a concentration of 50 mg/ml to 130 mg/ml. In some embodiments, the protein is present at a concentration of 110 mg/ml to 130 mg/ml.

In some embodiments, the composition further comprises at least one buffer, at least one stabilizer, and/or at least one surfactant. In some embodiments, the composition is liquid. In some embodiments, the composition is formulated for subcutaneous injection. In some embodiments, the composition is sterile. In some embodiments, the composition further comprises histidine HCl, trehalose dehydrate, methionine and/or polysorbate. In some embodiments, the composition comprises 110 to 130 mg/ml VEGF-A-binding protein, 10 mM to 30 mM histidine HCl, 200 mM to 220 mM trehalose dehydrate, 5 mM to 15 mM methionine, and/or 0.04% to 0.08% polysorbate 80. In some embodiments, the composition comprises 110 to 130 mg/ml HER2-binding protein, 10 mM to 30 mM histidine HCl, 200 mM to 220 mM trehalose dehydrate, 5 mM to 15 mM methionine, and/or 0.04% to 0.08% polysorbate 80.

Some embodiments provide a method of treating a condition in a subject in need thereof, comprising administering to the subject a therapeutically effective amount of the composition of the present disclosure.

In some embodiments, the condition is cancer. The cancer may be, for example, colorectal cancer, lung cancer, breast cancer, glioblastoma, kidney cancer, or ovarian cancer.

Some embodiments provide a nucleic acid encoding the VEGF-A-binding protein of the present disclosure. Some embodiments provide a nucleic acid encoding the HER2-binding protein of the present disclosure. Some embodiments provide a vector comprising a nucleic acid of the present disclosure. Some embodiments provide an expression cassette comprising a nucleic acid of the present disclosure. Some embodiments provide a host cell comprising a nucleic acid, a vector or an expression cassette of the present disclosure. Some embodiments provide a method of producing an antibody, comprising culturing a host cell of the present disclosure.

Some embodiments provide a kit comprising a container and the VEGF-A-binding protein of the present disclosure or the composition of the present disclosure. Some embodiments provide a kit comprising a container and the HER2-binding protein of the present disclosure or the composition of the present disclosure. In some embodiments, the container is glass or plastic. In some embodiments, the container is a vial or a syringe. In some embodiments, the kit further comprises a package insert or label indicating that the protein or composition can be used to treat cancer characterized by the overexpression or overactivity of VEGF-A. In some embodiments, the kit further comprises a package insert or label indicating that the protein or composition can be used to treat cancer characterized by the overexpression or overactivity of HER2. In some embodiments, the composition is provided at a volume of less than 2 ml or is 2 ml. In some embodiments, the composition is provided at a volume of 1.5 ml. In some embodiments, the composition is provided at a volume of 1 ml to 2.5 ml. In some embodiments, the composition is provided at a volume of 1 ml to 2 ml.

These and other embodiments of the present disclosure will be described in greater detail herein.

### BRIEF DESCRIPTION OF DRAWINGS

FIG. 1 shows a stability comparison of bevacizumab wild type (WT) and variants by SEC-HPLC. Monomer loss for WT and hyperglycosylated variants (50 mg/mL in histidine buffer, pH 6.0) was measured at various time points upon heat stress at 52° C. for 48 h. Data are the mean±SD. (n=3 experiments with three different protein batches *n=2 experiments with two different protein batches).

FIG. 2 shows a competitive ELISA of WT bevacizumab and single variants for VEGF-A. Bevacizumab and engineered proteins binding to VEGF were measured using a VEGF Human ELISA Kit in which the VEGF protein (1500 pg/mL) was pre-incubated and shaken for 60 min at 37° C. with bevacizumab or the variants at various concentrations (0.5 pM to 1 μM). The pre-mixes were then used in the ELISA, which allows the quantification of antibodies bound to the VEGF by reading absorbance at 450 nm. The more bevacizumab (or variants) binds to VEGF, the less VEGF can bind to the anti-VEGF IgG1 adhered onto the ELISA plate, and the lower the signal at 450 nm. Each assay was run at least in duplicate. The WT and variants bound to the target VEGF with the same affinity, as previously reported for bevacizumab.

### DETAILED DESCRIPTION

The aggregation of biotherapeutics is a major hindrance to the development of successful drug candidates; however, the propensity to aggregate is often identified too late in the development phase to permit modification to the protein's sequence. Incorporating rational design for the stability of proteins in early discovery has numerous benefits. Provided herein are drug candidate proteins lacking aggregation-prone regions. For example, the present disclosure provides a therapeutic monoclonal antibody, bevacizumab, lacking certain aggregation-prone regions on the Fab domain. The methods provided herein resulted in an up to four-fold reduction in monomer loss. Surprisingly, the mutations and modified glycosylation patterns of the Fab domain of the antibodies provided herein do not modify binding to the target. Thus, provided herein are therapeutic proteins with increased stability against aggregation.

Some embodiments provide Vascular Endothelial Growth Factor-A (VEGF-A)-binding proteins that have a reduced tendency to aggregate (e.g., are more stable) as compared to existing proteins. Other embodiments provide Human Epidermal Growth Factor Receptor 2 (HER2)-binding proteins that have a reduced tendency to aggregate as compared to existing proteins. Some embodiments provide compositions comprising the proteins and methods of use.

### Proteins

VEGF-A-Binding Proteins

The present disclosure provides VEGF-A-binding proteins that are less prone to aggregation as compared to existing proteins.

Provided herein, in some embodiments, are proteins that may selectively (preferentially) bind to cell antigen VEGF-A (i.e., VEGF-A antigen) and are referred to as “VEGF-A-binding proteins.” A protein (e.g., anti-VEGF-A antibody) selectively binds to an antigen (e.g., VEGF-A) if the protein binds or is capable of binding to the antigen with a greater affinity than the affinity with which the protein might bind to other proteins (e.g., proteins other than VEGF-A). Such binding may be measured or determined by standard protein-protein interaction assays (e.g., antibody-antigen or ligand-receptor assays) such as, for example, competitive assays, saturation assays, or standard immunoassays including, without limitation, enzyme-linked immunosorbent assays, radioimmunoassays and radio-immuno-filter binding assays.

VEGF-A protein is a growth factor active in angiogenesis, vasculogenesis and endothelial cell growth. It induces endothelial cell proliferation, promotes cell migration, inhibits apoptosis, and induces permeabilization of blood vessels. VEGF-A binds to the VEGFR1/Flt-1 and to VEGFR2/Kdr receptors, heparan sulfate and heparin. In some embodiments, the VEGF-A antigen may be human VEGF-A antigen. An example of an amino acid sequence of a human VEGF-A antigen is represented as GenBank Protein ID AAA35789.1 (SEQ ID NO: 8).

In some embodiments, a VEGF-A-binding protein may comprise a Fab heavy chain domain having an amino acid sequence modified at position 118 (L₁₁₈). For example, a VEGF-A-binding protein may comprise a Fab heavy chain domain having an amino acid sequence modified at position 118 from leucine (L) to asparagine (N) (SEQ ID NO: 4).

In some embodiments, a VEGF-A-binding protein may comprise a light chain domain having an amino acid sequence modified at position 160 (Q₁₆₀). For example, a VEGF-A-binding protein may comprise a light chain domain having an amino acid sequence modified at position 160 from glutamine (Q) to asparagine (N) (SEQ ID NO: 6). As another example, a VEGF-A-binding protein may comprise a light chain domain having an amino acid sequence modified at position 160 from glutamine (Q) to serine (S) (SEQ ID NO: 7).

In some embodiments, a VEGF-A-binding protein may comprise a light chain domain having an amino acid sequence modified at position 195 (E₁₉₅). For example, a VEGF-A-binding protein may comprise a light chain domain having an amino acid sequence modified at position 195 from glutamic acid (E) to asparagine (N) (SEQ ID NO: 5).

In some embodiments, a VEGF-A-binding protein may comprise a light chain domain having an amino acid sequence modified at position 107 (K₁₀₇). For example, a VEGF-A-binding protein may comprise a light chain domain having an amino acid sequence modified at position 107 from lysine (K) to asparagine (N) (SEQ ID NO: 12).

In some embodiments, a VEGF-A-binding protein may comprise a light chain domain having an amino acid sequence modified at position 140 (Y₁₄₀). For example, a VEGF-A-binding protein may comprise a light chain domain having an amino acid sequence modified at position 140 from tyrosine (Y) to serine (S) (SEQ ID NO: 13).

In some embodiments, a VEGF-A-binding protein may comprise a light chain domain having an amino acid sequence modified at position 169 (K₁₆₉). For example, a VEGF-A-binding protein may comprise a light chain domain having an amino acid sequence modified at position 169 from lysine (K) to asparagine (N) (SEQ ID NO: 14).

In some embodiments, a VEGF-A-binding protein may comprise a light chain domain having an amino acid sequence modified at position 170 (D₁₇₀). For example, a VEGF-A-binding protein may comprise a light chain domain having an amino acid sequence modified at position 170 from aspartic acid (D) to asparagine (N) (SEQ ID NO: 15).

In some embodiments, a VEGF-A-binding protein may comprise a Fab heavy chain domain having an amino acid sequence modified at position 118 (L₁₁₈) and a non-modified light chain domain. For example, a VEGF-A-binding protein may comprise a Fab heavy chain domain having an amino acid sequence modified at position 118 from L to N (SEQ ID NO: 4) and a non-modified light chain domain (SEQ ID NO: 1).

In some embodiments, a VEGF-A-binding protein may comprise a Fab heavy chain domain having an amino acid sequence modified at position 118 (L₁₁₈) and a light chain domain having an amino acid sequence modified at position 160 (Q₁₆₀). For example, a VEGF-A-binding protein may comprise a Fab heavy chain domain having an amino acid sequence modified at position 118 from L to N (SEQ ID NO: 4) and a light chain domain having an amino acid sequence modified at position 160 from Q to N (SEQ ID NO: 6), or modified at position 160 from Q to S (SEQ ID NO: 7).

In some embodiments, a VEGF-A-binding protein may comprise a Fab heavy chain domain having an amino acid sequence modified at position 118 (L₁₁₈) and a light chain domain having an amino acid sequence modified at position 195 (E₁₉₅). For example, a VEGF-A-binding protein may comprise a Fab heavy chain domain having an amino acid sequence modified at position 118 from L to N (SEQ ID NO: 4) and a light chain domain having an amino acid sequence modified at position 195 from E to N (SEQ ID NO: 5).

In some embodiments, a VEGF-A-binding protein may comprise a Fab heavy chain domain having an amino acid sequence modified at position 118 (L₁₁₈) and a light chain domain having an amino acid sequence modified at position 107 (K₁₀₇). For example, a VEGF-A-binding protein may comprise a Fab heavy chain domain having an amino acid sequence modified at position 118 from L to N (SEQ ID NO: 4) and a light chain domain having an amino acid sequence modified at position 107 from K to N (SEQ ID NO: 12).

In some embodiments, a VEGF-A-binding protein may comprise a Fab heavy chain domain having an amino acid sequence modified at position 118 (L₁₁₈) and a light chain domain having an amino acid sequence modified at position 140 (Y₁₄₀). For example, a VEGF-A-binding protein may comprise a Fab heavy chain domain having an amino acid sequence modified at position 118 from L to N (SEQ ID NO: 4) and a light chain domain having an amino acid sequence modified at position 140 from Y to S (SEQ ID NO: 13).

In some embodiments, a VEGF-A-binding protein may comprise a Fab heavy chain domain having an amino acid sequence modified at position 118 (L₁₁₈) and a light chain domain having an amino acid sequence modified at position 169 (K₁₆₉). For example, a VEGF-A-binding protein may comprise a Fab heavy chain domain having an amino acid sequence modified at position 118 from L to N (SEQ ID NO: 4) and a light chain domain having an amino acid sequence modified at position 169 from K to N (SEQ ID NO: 14).

In some embodiments, a VEGF-A-binding protein may comprise a Fab heavy chain domain having an amino acid sequence modified at position 118 (L₁₁₈) and a light chain domain having an amino acid sequence modified at position 170 (D₁₇₀). For example, a VEGF-A-binding protein may comprise a Fab heavy chain domain having an amino acid sequence modified at position 118 from L to N (SEQ ID NO: 4) and a light chain domain having an amino acid sequence modified at position 170 from D to N (SEQ ID NO: 15).

In some embodiments, a VEGF-A-binding protein may comprise a non-modified Fab heavy chain domain and a light chain domain having an amino acid sequence modified at position 160 (Q₁₆₀). For example, a VEGF-A-binding protein may comprise a non-modified Fab heavy chain domain (SEQ ID NO: 2) and a light chain domain having an amino acid sequence modified at position 160 from Q to N (SEQ ID NO: 6), or modified at position 160 from Q to S (SEQ ID NO: 7).

In some embodiments, a VEGF-A-binding protein may comprise a non-modified Fab heavy chain domain and a light chain domain having an amino acid sequence modified at position 195 (E₁₉₅). For example, a VEGF-A-binding protein may comprise a non-modified Fab heavy chain domain (SEQ ID NO: 2) and a light chain domain having an amino acid sequence modified at position 195 from E to N (SEQ ID NO: 5).

In some embodiments, a VEGF-A-binding protein may comprise a non-modified Fab heavy chain domain and a light chain domain having an amino acid sequence modified at position 107 (K₁₀₇). For example, a VEGF-A-binding protein may comprise a non-modified Fab heavy chain domain (SEQ ID NO: 2) and a light chain domain having an amino acid sequence modified at position 107 from K to N (SEQ ID NO: 12).

In some embodiments, a VEGF-A-binding protein may comprise a non-modified Fab heavy chain domain and a light chain domain having an amino acid sequence modified at position 140 (Y₁₄₀). For example, a VEGF-A-binding protein may comprise a non-modified Fab heavy chain domain (SEQ ID NO: 2) and a light chain domain having an amino acid sequence modified at position 140 from Y to S (SEQ ID NO: 13).

In some embodiments, a VEGF-A-binding protein may comprise a non-modified Fab heavy chain domain and a light chain domain having an amino acid sequence modified at position 169 (K₁₆₉). For example, a VEGF-A-binding protein may comprise a non-modified Fab heavy chain domain (SEQ ID NO: 2) and a light chain domain having an amino acid sequence modified at position 169 from K to N (SEQ ID NO: 14).

In some embodiments, a VEGF-A-binding protein may comprise a non-modified Fab heavy chain domain and a light chain domain having an amino acid sequence modified at position 170 (D₁₇₀). For example, a VEGF-A-binding protein may comprise a non-modified Fab heavy chain domain (SEQ ID NO: 2) and a light chain domain having an amino acid sequence modified at position 170 from D to N (SEQ ID NO: 15).

HER2-Binding Proteins

The present disclosure provides HER2-binding proteins that are less prone to aggregation as compared to existing proteins.

Provided herein, in some embodiments, are proteins that may selectively (preferentially) bind to cell antigen HER2 (i.e., HER2 antigen) and are referred to as “HER2-binding proteins.” HER2 protein has a molecular weight of approximately 138 kD. It is involved in transmembrane receptor protein tyrosine kinase activity. In some embodiments, the HER2 antigen may be human HER2 antigen. An example of an amino acid sequence of a human HER2 antigen is represented as NCBI Reference Sequence NP_004439.2 (SEQ ID NO: 26).

In some embodiments, a HER2-binding protein may comprise a Fab heavy chain domain having an amino acid sequence modified at position 115 (L₁₁₅). For example, a HER2-binding protein may comprise a Fab heavy chain domain having an amino acid sequence modified at position 115 from leucine (L) to asparagine (N) (SEQ ID NO: 23).

In some embodiments, a HER2-binding protein may comprise a light chain domain having an amino acid sequence modified at position 160 (Q₁₆₀). For example, a HER2-binding protein may comprise a light chain domain having an amino acid sequence modified at position 160 from glutamine (Q) to asparagine (N) (SEQ ID NO: 24).

In some embodiments, a HER2-binding protein may comprise a light chain domain having an amino acid sequence modified at position 195 (E₁₉₅). For example, a HER2-binding protein may comprise a light chain domain having an amino acid sequence modified at position 195 from glutamic acid (E) to asparagine (N) (SEQ ID NO: 25).

In some embodiments, a HER2-binding protein may comprise a Fab heavy chain domain having an amino acid sequence modified at position 115 (L₁₁₅) and a non-modified light chain domain. For example, a HER2-binding protein may comprise a Fab heavy chain domain having an amino acid sequence modified at position 115 from L to N (SEQ ID NO: 23) and a non-modified light chain domain (SEQ ID NO: 20).

In some embodiments, a HER2-binding protein may comprise a Fab heavy chain domain having an amino acid sequence modified at position 115 (L₁₁₅) and a light chain domain having an amino acid sequence modified at position 160 (Q₁₆₀). For example, a HER2-binding protein may comprise a Fab heavy chain domain having an amino acid sequence modified at position 115 from L to N (SEQ ID NO: 23) and a light chain domain having an amino acid sequence modified at position 160 from Q to N (SEQ ID NO: 24).

In some embodiments, a HER2-binding protein may comprise a Fab heavy chain domain having an amino acid sequence modified at position 115 (L₁₁₅) and a light chain domain having an amino acid sequence modified at position 195 (E₁₉₅). For example, a HER2-binding protein may comprise a Fab heavy chain domain having an amino acid sequence modified at position 115 from L to N (SEQ ID NO: 23) and a light chain domain having an amino acid sequence modified at position 195 from E to N (SEQ ID NO: 25).

In some embodiments, a HER2-binding protein may comprise a non-modified Fab heavy chain domain and a light chain domain having an amino acid sequence modified at position 160 (Q₁₆₀). For example, a HER2-binding protein may comprise a non-modified Fab heavy chain domain (SEQ ID NO: 21) and a light chain domain having an amino acid sequence modified at position 160 from Q to N (SEQ ID NO: 24).

In some embodiments, a HER2-binding protein may comprise a non-modified Fab heavy chain domain and a light chain domain having an amino acid sequence modified at position 195 (E₁₉₅). For example, a HER2-binding protein may comprise a non-modified Fab heavy chain domain (SEQ ID NO: 21) and a light chain domain having an amino acid sequence modified at position 195 from E to N (SEQ ID NO: 25).

### Antibodies

In some embodiments, VEGF-A-binding proteins or HER2 binding proteins are antibodies. In some embodiments, the proteins are monoclonal antibodies such as, for example, chimeric, human or humanized monoclonal antibodies. In some embodiments, the anti-VEGF-A antibodies or anti-HER2 antibodies may be humanized monoclonal antibodies.

As used herein, the term “antibody” refers to a whole antibody. In some embodiments, the proteins provided herein may be antigen-binding fragments of an antibody, or single antibody chains. An antibody is a glycoprotein comprising at least two heavy (H) chains and two light (L) chains inter-connected by disulfide bonds. Each heavy chain is comprised of a heavy chain variable region (abbreviated herein as V_{H}) and a heavy chain constant region. The heavy chain constant region is comprised of three subdomains, C_{H1}, C_{H2} and C_{H3}. Each light chain is comprised of a light chain variable region (abbreviated herein as V_{L}) and a light chain constant region. The light chain constant region is comprised of one subdomain, C_{L}. The V_{H} and V_{L} regions can be further subdivided into regions of hypervariability, termed complementarity determining regions (CDR), interspersed with regions that are more conserved, termed framework regions (FR). Each V_{H} and V_{L} is composed of three CDRs and four FRs arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (Clq) of the classical complement system.

Protein, including antibodies, may be described in terms of proteolytic fragments including without limitation Fv, Fab, Fab′ and F(ab′)₂ fragments. Such fragments may be prepared by standard methods (see, e.g., Coligan et al. Current Protocols in Immunology, John Wiley & Sons, 1991-1997, incorporated herein by reference). An antibody may comprise at least three proteolytic fragments (i.e., fragments produced by cleavage with papain): two Fab fragments, each containing a light chain domain and a heavy chain domain (designated herein as a “Fab heavy chain domain”) and one Fc fragment containing two Fc domains. Each light chain domain contains a V_{L} and a C_{L} subdomain, each Fab heavy chain domain contains a V_{H} and a C_{H1} subdomain, and each Fc domain contains a C_{H2} and C_{H3} subdomain.

As used herein, the term “monoclonal antibody” refers to an antibody obtained from a single clonal population of immunoglobulins that bind to the same epitope of an antigen. Monoclonal antibodies have the same Ig gene rearrangement and thus demonstrate identical binding specificity. Methods for preparing monoclonal antibodies, as described herein, are known in the art. Monoclonal antibodies can be prepared by a variety of methods. For example, monoclonal antibodies may be made by a hybridoma method (see, e.g., Kohler et al., Nature, 1975, 256: 495, incorporated herein by reference), or may be made by recombinant DNA methods (see, e.g., U.S. Pat. No. 4,816,567, incorporated herein by reference). The monoclonal antibodies may also be isolated from phage antibody libraries. (See e.g., Clarkson et al., Nature, 1991, 352: 624-628 and Marks et al., J. Mol. Biol., 1991, 222: 581-597, incorporated herein by reference).

Human monoclonal antibodies may be made by any of the methods known in the art, including those disclosed in U.S. Pat. No. 5,567,610, U.S. Pat. No. 5,565,354, U.S. Pat. No. 5,571,893, Kozber, J. Immunol., 1984, 133: 3001, Brodeur, et al., Monoclonal Antibody Production Techniques and Applications, p. 51-63 (Marcel Dekker, Inc., new York, 1987), and Boerner et al., J. Immunol., 1991, 147: 86-95. Human antibodies may be obtained by recovering antibody-producing lymphocytes from the blood or other tissues of humans producing antibody to an antigen of interest (e.g., VEGF-A or HER2). These lymphocytes can be treated to produce cells that grow independently in the laboratory under appropriate culture conditions. The cell cultures can be screened for production of antibodies to the antigen of interest and then cloned. Clonal cultures can be used to produce human monoclonal antibodies to VEGF-A or HER2, or the genetic elements encoding the variable portions of the heavy and light chain of the antibodies can be cloned and inserted into nucleic acid vectors for production of antibodies of different types. In addition to the conventional methods for preparing human monoclonal antibodies, such antibodies may also be prepared by immunizing transgenic animals that are capable of producing human antibodies (e.g., Jakobovits, et al., PNAS USA, 1993, 90: 2551, Jakobovits, et al., Nature, 1993, 362: 255-258, Bruggermann, et al., Year in Immunol., 1993, 7:33 and U.S. Pat. No. 5,569,825).

As used herein, “humanized monoclonal antibody” refers to monoclonal antibodies having at least human constant regions and an antigen-binding region, such as one, two or three CDRs, from a non-human species. Humanized antibodies specifically recognize antigens of interest, but will not evoke an immune response in humans against the antibody itself. As an example, murine CDRs may grafted into the framework region of a human antibody to prepare the humanized antibody (e.g., L. Riechmann, et al., Nature, 1988, 332, 323, and M. S. Neuberger et al., Nature, 1985, 314, 268). Alternatively, humanized monoclonal antibodies may be constructed by replacing the non-CDR regions of non-human antibodies with similar regions of human antibodies while retaining the epitopic specificity of the original antibodies. For example, non-human CDRs and optionally some of the framework regions may be covalently joined to human FR and/or Fc/pFc′ regions to produce functional antibodies.

As used herein, the term “chimeric antibody” refers to a monoclonal antibody comprising a variable region from one source (e.g., species) and at least a portion of a constant region derived from a different source. In some embodiments, chimeric antibodies are prepared by recombinant DNA techniques. In some embodiments, the chimeric antibodies comprise a murine variable region and a human constant region. Such chimeric antibodies may, in some embodiments, be the product of expressed immunoglobulin genes comprising DNA segments encoding murine immunoglobulin variable regions and DNA segments encoding human immunoglobulin constant regions. Methods for producing chimeric antibodies involve conventional recombinant DNA and gene transfection techniques (see, e.g., Morrison, S. L., et al., Proc. Natl. Acad. Sci. USA, 1984, 81: 6851-6855; U.S. Pat. No. 5,202,238; and U.S. Pat. No. 5,204,244).

Antigen-binding antibody fragments are also encompassed by the present disclosure. Only a small portion of an antibody molecule, the paratope, is involved in the binding of the antibody to its epitope (see, in general, Clark, W. R. (1986) The Experimental Foundations of Modern Immunology Wiley & Sons, Inc., New York; Roitt, I. (1991) Essential Immunology, 7th Ed., Blackwell Scientific Publications, Oxford). The pFc′ and Fc regions of the antibody, for example, are effectors of the complement cascade but are not involved in antigen binding. An antibody from which the pFc′ region has been enzymatically cleaved, or which has been produced without the pFc′ region, designated an F(ab′)2 fragment, retains both of the antigen binding sites of an intact antibody. An isolated F(ab′)2 fragment is referred to as a bivalent monoclonal fragment because of its two antigen binding sites. Similarly, an antibody from which the Fc region has been enzymatically cleaved, or which has been produced without the Fc region, designated a Fab fragment, retains one of the antigen binding sites of an intact antibody molecule. Further, Fab fragments consist of a covalently bound antibody light chain and a portion of the antibody heavy chain denoted Fd (heavy chain variable region, referred to herein as Fab heavy chain domain). The Fd fragments are the major determinant of antibody specificity (a single Fd fragment may be associated with up to ten different light chains without altering antibody specificity) and Fd fragments retain epitope-binding ability in isolation.

The terms Fab, Fc, pFc′, F(ab′)2 and Fv are employed with either standard immunological meanings (Klein, Immunology (John Wiley, New York, N.Y., 1982); Clark, W. R. (1986) The Experimental Foundations of Modern Immunology (Wiley & Sons, Inc., New York); Roitt, I. (1991) Essential Immunology, 7th Ed., (Blackwell Scientific Publications, Oxford)). Well-known functionally active antibody fragments include but are not limited to F(ab′)2, Fab, Fv and Fd fragments of antibodies. These fragments, which lack the Fc fragment of intact antibody, clear more rapidly from the circulation, and may have less non-specific tissue binding than an intact antibody (Wahl et al., J. Nucl. Med. 24:316-325 (1983)). For example, single-chain antibodies may be constructed in accordance with the methods described in U.S. Pat. No. 4,946,778. Such single-chain antibodies include the variable regions of the light and heavy chains joined by a flexible linker moiety. Methods for obtaining a single domain antibody (“Fd”) which comprises an isolated variable heavy chain single domain, also have been reported (see, e.g., Ward, et al., Nature, 1989, 341:644-646, disclosing a method of screening to identify an antibody heavy chain variable region (V_{H} single domain antibody) with sufficient affinity for its target epitope to bind thereto in isolated form). Methods for making recombinant Fv fragments based on known antibody heavy chain and light chain variable region sequences are known in the art and have been described, (see, e.g., Moore et al., U.S. Pat. No. 4,462,334). Other references describing the use and generation of antibody fragments include, e.g., Fab fragments (Tijssen, Practice and Theory of Enzyme Immunoassays (Elsevieer, Amsterdam, 1985)), Fv fragments (Hochman, et al., Biochemistry, 1973, 12: 1130; Sharon, et al., Biochemistry, 1976, 15: 1591; Ehrilch, et al., U.S. Pat. No. 4,355,023) and portions of antibody molecules (Audilore-Hargreaves, U.S. Pat. No. 4,470,925). Thus, antibody fragments may be constructed from intact antibodies without destroying the specificity of the antibodies for the VEGF-A or HER2 epitope.

In some embodiments, VEGF-A-binding proteins or HER2-binding proteins are recombinant forms of antibodies. In some embodiments, the proteins may be stabilized Fv fragments having single chain Fv forms (e.g., scFv) and comprising a peptide linker joining the variable heavy chain and variable light chain domains. In other embodiments, the proteins may be Fv fragments stabilized by inter-chain disulfide linkage (e.g., dsFv). In some embodiments, the proteins may contain additional cysteine residues engineered to facilitate the conjoining of the variable heavy chain and variable light chain domains. In further embodiments, the proteins may be minibodies or single variable domain antibodies (“dAbs”). Minibodies may be genetically engineered antigen-binding constructs having structures reminiscent of antibodies (e.g., having Fab and/or Fc regions with a reduced number of variable and/or constant domains). In other embodiments, the proteins may include dimerization domains (e.g. “leucine zippers”) or other chemical modifications.

In some embodiments, the antibodies (e.g., anti-VEGF-A antibodies or anti-HER2 antibodies) may exhibit an affinity for their target that is similar to (or greater than) the affinity exhibited by existing antibodies that bind to the same target. As an example, anti-VEGF-A antibodies recognize and bind to VEGF-A on cells with an affinity at least comparable to that of bevacizumab. As another example, anti-HER2 antibodies recognize and bind to HER2 on cells with an affinity at least comparable to that of trastuzumab.

### Hyperglycosylated Proteins

Glycosylation refers to the reaction in which a carbohydrate (a glycosyl donor) is attached to a hydroxyl or other functional group of another molecule (a glycosyl acceptor). Glycosylation refers to the enzymatic process that attaches a glycan to a protein. For example, a protein that contains an N-linked carbohydrate is considered glycosylated. In some embodiments, a variant protein herein is considered to be “hyperglycosylated” if it contains at least one more carbohydrate relative to a wild-type protein (e.g., non-modified bevacizumab or non-modified trastuzumab). In some embodiments, a variant protein herein is considered to be “hyperglycosylated” if it contains at least two more carbohydrate relative to a wild-type protein. In some embodiments, a variant protein herein is considered to be “hyperglycosylated” if it contains at least three, at least four, or at least five more carbohydrates relative to a wild-type protein

Hyperglycosylated VEGF-A-Binding Proteins

Some embodiments provide hyperglycosylated forms of the VEGF-A-binding proteins of the present disclosure.

Thus, some embodiments provide a hyperglycosylated VEGF-A-binding protein comprising a Fab heavy chain domain having an amino acid sequence modified at position 118 (L₁₁₈). For example, a hyperglycosylated VEGF-A-binding protein may comprise a Fab heavy chain domain having an amino acid sequence modified at position 118 from leucine (L) to asparagine (N) (SEQ ID NO: 4).

In some embodiments, a hyperglycosylated VEGF-A-binding protein may comprise a light chain domain having an amino acid sequence modified at position 195 (E₁₉₅). For example, a hyperglycosylated VEGF-A-binding protein may comprise a light chain domain having an amino acid sequence modified at position 195 from glutamic acid (E) to asparagine (N) (SEQ ID NO: 5).

In some embodiments, a hyperglycosylated VEGF-A-binding protein may comprise a light chain domain having an amino acid sequence modified at position 160 (Q₁₆₀). For example, a hyperglycosylated VEGF-A-binding protein may comprise a light chain domain having an amino acid sequence modified at position 160 from glutamine (Q) to asparagine (N) (SEQ ID NO: 6). As another example, a hyperglycosylated VEGF-A-binding protein may comprise a light chain domain having an amino acid sequence modified at position 160 from glutamine (Q) to serine (S) (SEQ ID NO: 7).

In some embodiments, a hyperglycosylated VEGF-A-binding protein may comprise a light chain domain having an amino acid sequence modified at position 107 (K₁₀₇). For example, a VEGF-A-binding protein may comprise a light chain domain having an amino acid sequence modified at position 107 from lysine (K) to asparagine (N) (SEQ ID NO: 12).

In some embodiments, a hyperglycosylated VEGF-A-binding protein may comprise a light chain domain having an amino acid sequence modified at position 140 (Y₁₄₀). For example, a VEGF-A-binding protein may comprise a light chain domain having an amino acid sequence modified at position 140 from tyrosine (Y) to serine (S) (SEQ ID NO: 13).

In some embodiments, a hyperglycosylated VEGF-A-binding protein may comprise a light chain domain having an amino acid sequence modified at position 169 (K₁₆₉). For example, a VEGF-A-binding protein may comprise a light chain domain having an amino acid sequence modified at position 169 from lysine (L) to asparagine (N) (SEQ ID NO: 14).

In some embodiments, a hyperglycosylated VEGF-A-binding protein may comprise a light chain domain having an amino acid sequence modified at position 170 (D₁₇₀). For example, a VEGF-A-binding protein may comprise a light chain domain having an amino acid sequence modified at position 195 from aspartic acid (D) to asparagine (N) (SEQ ID NO: 15).

In some embodiments, a hyperglycosylated VEGF-A-binding protein may comprise a Fab heavy chain domain having an amino acid sequence modified at position 118 (L₁₁₈) and a non-modified light chain domain. For example, a hyperglycosylated VEGF-A-binding protein may comprise a Fab heavy chain domain having an amino acid sequence modified at position 118 from L to N (SEQ ID NO: 4) and a non-modified light chain domain (SEQ ID NO: 1).

In some embodiments, a hyperglycosylated VEGF-A-binding protein may comprise a Fab heavy chain domain having an amino acid sequence modified at position 118 (L₁₁₈) and a light chain domain having an amino acid sequence modified at position 195 (E₁₉₅). For example, a hyperglycosylated VEGF-A-binding protein may comprise a Fab heavy chain domain having an amino acid sequence modified at position 118 from L to N (SEQ ID NO: 4) and a light chain domain having an amino acid sequence modified at position 195 from E to N (SEQ ID NO: 5).

In some embodiments, a hyperglycosylated VEGF-A-binding protein may comprise a Fab heavy chain domain having an amino acid sequence modified at position 118 (L₁₁₈) and a light chain domain having an amino acid sequence modified at position 160 (Q₁₆₀). For example, a hyperglycosylated VEGF-A-binding protein may comprise a Fab heavy chain domain having an amino acid sequence modified at position 118 from L to N (SEQ ID NO: 4) and a light chain domain having an amino acid sequence modified at position 160 from Q to N (SEQ ID NO: 6), or modified at position 160 from Q to S (SEQ ID NO: 7).

In some embodiments, a hyperglycosylated VEGF-A-binding protein may comprise a Fab heavy chain domain having an amino acid sequence modified at position 118 (L₁₁₈) and a light chain domain having an amino acid sequence modified at position 107 (K₁₀₇). For example, a VEGF-A-binding protein may comprise a Fab heavy chain domain having an amino acid sequence modified at position 118 from L to N (SEQ ID NO: 4) and a light chain domain having an amino acid sequence modified at position 107 from K to N (SEQ ID NO: 12).

In some embodiments, a hyperglycosylated VEGF-A-binding protein may comprise a

Fab heavy chain domain having an amino acid sequence modified at position 118 (L₁₁₈) and a light chain domain having an amino acid sequence modified at position 140 (Y₁₄₀). For example, a VEGF-A-binding protein may comprise a Fab heavy chain domain having an amino acid sequence modified at position 118 from L to N (SEQ ID NO: 4) and a light chain domain having an amino acid sequence modified at position 140 from Y to S (SEQ ID NO: 13).

In some embodiments, a hyperglycosylated VEGF-A-binding protein may comprise a Fab heavy chain domain having an amino acid sequence modified at position 118 (L₁₁₈) and a light chain domain having an amino acid sequence modified at position 169 (K₁₆₉). For example, a VEGF-A-binding protein may comprise a Fab heavy chain domain having an amino acid sequence modified at position 118 from L to N (SEQ ID NO: 4) and a light chain domain having an amino acid sequence modified at position 169 from K to N (SEQ ID NO: 14).

In some embodiments, a hyperglycosylated VEGF-A-binding protein may comprise a Fab heavy chain domain having an amino acid sequence modified at position 118 (L₁₁₈) and a light chain domain having an amino acid sequence modified at position 170 (D₁₇₀). For example, a VEGF-A-binding protein may comprise a Fab heavy chain domain having an amino acid sequence modified at position 118 from L to N (SEQ ID NO: 4) and a light chain domain having an amino acid sequence modified at position 170 from D to N (SEQ ID NO: 15).

In some embodiments, a hyperglycosylated VEGF-A-binding protein may comprise a non-modified Fab heavy chain domain and a light chain domain having an amino acid sequence modified at position 195 (E₁₉₅). For example, a hyperglycosylated VEGF-A-binding protein may comprise a non-modified Fab heavy chain domain (SEQ ID NO: 2) and a light chain domain having an amino acid sequence modified at position 195 from E to N (SEQ ID NO: 5).

In some embodiments, a hyperglycosylated VEGF-A-binding protein may comprise a non-modified Fab heavy chain domain and a light chain domain having an amino acid sequence modified at position 160 (Q₁₆₀). For example, a hyperglycosylated VEGF-A-binding protein may comprise a non-modified Fab heavy chain domain (SEQ ID NO: 2) and a light chain domain having an amino acid sequence modified at position 160 from Q to N (SEQ ID NO: 6), or modified at position 160 from Q to S (SEQ ID NO: 7).

In some embodiments, a hyperglycosylated VEGF-A-binding protein may comprise a non-modified Fab heavy chain domain and a light chain domain having an amino acid sequence modified at position 107 (K₁₀₇). For example, a VEGF-A-binding protein may comprise a non-modified Fab heavy chain domain (SEQ ID NO: 2) and a light chain domain having an amino acid sequence modified at position 107 from K to N (SEQ ID NO: 12).

In some embodiments, a hyperglycosylated VEGF-A-binding protein may comprise a non-modified Fab heavy chain domain and a light chain domain having an amino acid sequence modified at position 140 (Y₁₄₀). For example, a VEGF-A-binding protein may comprise a non-modified Fab heavy chain domain (SEQ ID NO: 2) and a light chain domain having an amino acid sequence modified at position 140 from Y to S (SEQ ID NO: 13).

In some embodiments, a hyperglycosylated VEGF-A-binding protein may comprise a non-modified Fab heavy chain domain and a light chain domain having an amino acid sequence modified at position 169 (K₁₆₉). For example, a VEGF-A-binding protein may comprise a non-modified Fab heavy chain domain (SEQ ID NO: 2) and a light chain domain having an amino acid sequence modified at position 169 from K to N (SEQ ID NO: 14).

In some embodiments, a hyperglycosylated VEGF-A-binding protein may comprise a non-modified Fab heavy chain domain and a light chain domain having an amino acid sequence modified at position 170 (D₁₇₀). For example, a VEGF-A-binding protein may comprise a non-modified Fab heavy chain domain (SEQ ID NO: 2) and a light chain domain having an amino acid sequence modified at position 170 from D to N (SEQ ID NO: 15).

Hyperglycosylated HER2-Binding Proteins

Some embodiments provide hyperglycosylated forms of the HER2-binding proteins of the present disclosure.

Thus, some embodiments provide a hyperglycosylated HER2-binding protein comprising a Fab heavy chain domain having an amino acid sequence modified at position 115 (L₁₁₅). For example, a HER2-binding protein may comprise a Fab heavy chain domain having an amino acid sequence modified at position 115 from leucine (L) to asparagine (N) (SEQ ID NO: 23).

In some embodiments, a hyperglycosylated HER2-binding protein may comprise a light chain domain having an amino acid sequence modified at position 160 (Q₁₆₀). For example, a HER2-binding protein may comprise a light chain domain having an amino acid sequence modified at position 160 from glutamine (Q) to asparagine (N) (SEQ ID NO: 24).

In some embodiments, a hyperglycosylated HER2-binding protein may comprise a light chain domain having an amino acid sequence modified at position 195 (E₁₉₅). For example, a HER2-binding protein may comprise a light chain domain having an amino acid sequence modified at position 195 from glutamic acid (E) to asparagine (N) (SEQ ID NO: 25).

In some embodiments, a hyperglycosylated HER2-binding protein may comprise a Fab heavy chain domain having an amino acid sequence modified at position 115 (L₁₁₅) and a non-modified light chain domain. For example, a hyperglycosylated HER2-binding protein may comprise a Fab heavy chain domain having an amino acid sequence modified at position 115 from L to N (SEQ ID NO: 23) and a non-modified light chain domain (SEQ ID NO: 20).

In some embodiments, a hyperglycosylated HER2-binding protein may comprise a Fab heavy chain domain having an amino acid sequence modified at position 115 (L₁₁₅) and a light chain domain having an amino acid sequence modified at position 160 (Q₁₆₀). For example, a HER2-binding protein may comprise a Fab heavy chain domain having an amino acid sequence modified at position 115 from L to N (SEQ ID NO: 23) and a light chain domain having an amino acid sequence modified at position 160 from Q to N (SEQ ID NO: 24).

In some embodiments, a hyperglycosylated HER2-binding protein may comprise a Fab heavy chain domain having an amino acid sequence modified at position 115 (L₁₁₅) and a light chain domain having an amino acid sequence modified at position 195 (E₁₉₅). For example, a HER2-binding protein may comprise a Fab heavy chain domain having an amino acid sequence modified at position 115 from L to N (SEQ ID NO: 23) and a light chain domain having an amino acid sequence modified at position 195 from E to N (SEQ ID NO: 25).

In some embodiments, a hyperglycosylated HER2-binding protein may comprise a non-modified Fab heavy chain domain and a light chain domain having an amino acid sequence modified at position 160 (Q₁₆₀). For example, a HER2-binding protein may comprise a non-modified Fab heavy chain domain (SEQ ID NO: 21) and a light chain domain having an amino acid sequence modified at position 160 from Q to N (SEQ ID NO: 24).

In some embodiments, a hyperglycosylated HER2-binding protein may comprise a non-modified Fab heavy chain domain and a light chain domain having an amino acid sequence modified at position 195 (E₁₉₅). For example, a HER2-binding protein may comprise a non-modified Fab heavy chain domain (SEQ ID NO: 21) and a light chain domain having an amino acid sequence modified at position 195 from E to N (SEQ ID NO: 25).

### Nucleic Acids

Some embodiments provide nucleic acids that encode VEGF-A-binding proteins or HER2-bindingn proteins. As used herein, the term “nucleic acid” refers to single-stranded and double-stranded nucleic acids and ribonucleic acids as well as deoxyribonucleic acids. In some embodiments, the nucleic acids are double-stranded DNA such as cDNA or single-stranded RNA such as mRNA. Nucleic acids may comprise naturally occurring and/or synthetic nucleotides and can be naturally or synthetically modified, for example by methylation, 5′- and/or 3′-capping. The sequence of the nucleic acids may have any nucleotide sequence suitable for encoding proteins. The nucleic acids may be one consecutive nucleic acid molecule or they may be composed of several nucleic acid molecules, each coding for a different part of the protein. In some embodiments, the nucleic acid sequences may be at least partially adapted to a specific codon usage, for example, human codon usage, of the host cells or organisms in which the nucleic acids are to be expressed. The nucleic acids may be double-stranded or single-stranded DNA or RNA.

If antibodies are single chain constructs, the nucleic acids encoding them may be single nucleic acid molecules containing a coding region which codes for the entire antibody. If antibodies are composed of more than one amino acid chain, the nucleic acids may be, for example, single nucleic acid molecules containing several coding regions each coding for one of the amino acid chains of the antibodies. In some embodiments, the coding regions may be separated by regulatory elements such as IRES elements in order to generate separate amino acid chains. In some embodiments, the nucleic acids may be composed of several nucleic acid molecules wherein each nucleic acid molecule may comprise one or more coding regions, each coding for one of the amino acid chains of the antibodies. The nucleic acids may also comprise additional nucleic acid sequences or other modifications which, for example, may code for other proteins, may influence the transcription and/or translation of the coding region(s), may influence the stability or other physical or chemical properties of the nucleic acid, or may have no function at all.

### Conjugates

In some embodiments, VEGF-A-binding proteins may be a conjugate. In some embodiments, HER2-binding proteins may be a conjugate. As used herein, the term “conjugate” refers two or more compounds which are linked together so that at least some of the properties from each compound are retained in the conjugate. Linking may be achieved by a covalent or non-covalent bond. In some embodiments, the compounds of the conjugate are linked via a covalent bond. The different compounds of a conjugate may be directly bound to each other via one or more covalent bonds between atoms of the compounds. Alternatively, the compounds may be bound to each other via a linker molecule wherein the linker is covalently attached to atoms of the compounds. If the conjugate is composed of more than two compounds, then these compounds may be, for example, linked in a chain conformation, one compound attached to the next compound, or several compounds each may be attached to one central compound.

Conjugates may comprise additional agents that are useful in therapy, diagnosis, prognosis and/or monitoring of a condition such as, for example, cancer. Examples of additional agents include, without limitation, antibodies or fragments of antibodies, enzymes, interaction domains, stabilizing domains, signaling sequences, detectable labels, fluorescent dyes, toxins, catalytic antibodies, cytolytic components, immunomodulators, immunoeffectors, MHC class I or class II antigens, chelators for radioactive labeling, radioisotopes, liposomes, transmembrane domains, viruses, and cells. In some embodiments, the additional agents are radionuclides or a cytotoxic agents capable of killing cells (e.g., cancer cells), such as chemotherapeutic agents. Examples of other agents that may be used include alkylating agents such as cisplatin, anti-metabolites, plant alkaloids and terpenoids, vinca alkaloids, podophyllotoxin, taxanes such as taxol, topoisomerase inhibitors such as irinotecan and topotecan, and/or antineoplastics such as doxorubicin.

### Vectors and Expression Cassettes

Some embodiments vectors and/or expression cassettes comprising proteins as provided herein. The term “vector” refers to any intermediary vehicle for a nucleic acid that enables the nucleic acid, for example, to be introduced into prokaryotic and/or eukaryotic cells and, in some instances, to be integrated into a genome. Vectors of this kind may be replicated and/or expressed in the cells. Vectors may comprise plasmids, phagemids, bacteriophages or viral genomes. The term “plasmid,” as used herein, may refer to a construct of extrachromosomal genetic material, for example, a circular DNA duplex, which can replicate independently of chromosomal DNA.

The term “expression cassette” refers to nucleic acid constructs that are capable of enabling and regulating the expression of coding nucleic acid sequences introduced therein. Expression cassettes may comprise promoters, ribosome binding sites, enhancers and/or other control elements which regulate transcription of a gene or translation of an mRNA. The exact structure of expression cassettes may vary as a function of the species or cell type, but generally comprises 5′-untranscribed and 5′-and 3′-untranslated sequences which are involved in initiation of transcription and translation, respectively, such as TATA box, capping sequence, CAAT sequence, and the like. More specifically, 5′-untranscribed expression control sequences may comprise a promoter region, which includes a promoter sequence for transcriptional control of operatively connected nucleic acids. Expression cassettes may also comprise enhancer sequences or upstream activator sequences.

The term “promoter” refers to nucleic acid sequences which are located upstream (5′) of the nucleic acid sequences which are to be expressed and control expression of the sequence by providing a recognition and binding site for RNA-polymerases. Promoters may include further recognition and binding sites for additional factors which may be involved in the regulation of gene transcription. Promoters may control the transcription of a prokaryotic or eukaryotic gene. Furthermore, promoters may be inducible, i.e., initiate transcription in response to an inducing agent, or may be constitutive if transcription is not controlled by an inducing agent. Genes that are under the control of inducible promoters are not expressed or are only expressed to a small extent if inducing agents are absent. In the presence of inducing agents the genes are switched on or the level of transcription is increased. This is mediated, in general, by binding of a specific transcription factor.

In addition, the expression cassettes or vectors may comprise other elements, for example, elements which may influence and/or regulate transcription and/or translation of the nucleic acids, amplification and/or reproduction of the expression cassettes or vectors, integration of the expression cassettes or vectors into the genome of host cells, and/or copy number of the expression cassettes or vectors in host cells. Suitable expression cassettes and vectors comprising respective expression cassettes for expressing proteins (e.g., antibodies) are well known.

### Host Cells

In some embodiments, VEGF-A binding proteins are produced by the host cells or cell lines as described above. In some embodiments, HER2 binding proteins are produced by the host cells or cell lines as described above.

Some embodiments provide host cells comprising the nucleic acids provided herein or the expression cassettes or vectors provided herein. The nucleic acids may be present in the form of a single copy or of two or more copies and, in some embodiments, are expressed in the host cells. The term “host cell” refers to any cell which can be transformed or transfected with an exogenous nucleic acid. They may be isolated cells or cells comprised in a tissue. The cells may be derived from a multiplicity of tissue types and may comprise primary cells and cell lines. Host cells may be prokaryotic (e.g., Escherichia coli) or eukaryotic (e.g., mammalian, in particular human, yeast and/or insect).

In some embodiments, host cells are bacterial cells such as E. coli, yeast cells such as a Saccharomyces cells (e.g., S. cerevisiae), insect cells such as a Sf9 cells, or mammalian cells such as human cells, for example, tumor-derived human cells, hamster cells (e.g., Chinese Hamster Ovary cells), or primate cells. In some embodiments, the host cells are derived from human myeloid leukemia cells. Examples of cell lines for use herein include, without limitation, K562, KG1, MUTZ-3, NM-F9, NM-D4, NM-H9D8, NM-H9D8-E6, NM H9D8-E6Q12, GT-2X, and cells or cell lines derived from any of these host cells, or mixture of cells or cell lines comprising at least one of those cells. These cell lines and their properties are described in detail in the PCT application WO 2008/028686 A2.

In some embodiments, host cells are optimized for expression of glycoproteins, in particular antibodies, having a specific glycosylation pattern. In some embodiments, the codon usage in the coding region of the nucleic acids and/or the promoters and the additional elements of the expression cassettes or vectors are compatible with and, in some instances, optimized for the type of host cell used.

### Compositions

Some embodiments provide compositions comprising any of the VEGF-A-binding proteins or HER2-binding proteins, the nucleic acids, the expression cassettes and/or vectors, the host cells, or the conjugates, as described herein. The compositions may also contain more than one of these components. Furthermore, the compositions may comprise one or more additional components selected from buffers, solubilizers, surfactants, carriers, excipients, solvents, and/or diluents.

In some embodiments, compositions comprise a VEGF-A-binding protein comprising a heavy chain domain having an amino acid sequence of SEQ ID NO: 2, and a light chain domain having an amino acid sequence of SEQ ID NO: 5, SEQ ID NO: 6, SEQ ID NO: 7, SEQ NO NO: 12, SEQ ID NO: 13, SEQ ID NO: 14 or SEQ ID NO: 15. In some embodiments, the light chain domain has an amino acid sequence of SEQ ID NO: 5. In some embodiments, the light chain domain has an amino acid sequence of SEQ ID NO: 6. In some embodiments, the light chain domain has an amino acid sequence of SEQ ID NO: 7. In some embodiments, the light chain domain has an amino acid sequence of SEQ ID NO: 12. In some embodiments, the light chain domain has an amino acid sequence of SEQ ID NO: 13. In some embodiments, the light chain domain has an amino acid sequence of SEQ ID NO: 14. In some embodiments, the light chain domain has an amino acid sequence of SEQ ID NO: 15.

In some embodiments, compositions comprise a VEGF-A-binding protein comprising a heavy chain domain having an amino acid sequence of SEQ ID NO: 4, and a light chain domain having an amino acid sequence of SEQ ID NO: 1.

In some embodiments, compositions comprise a VEGF-A-binding protein comprising a heavy chain domain having an amino acid sequence of SEQ ID NO: 4, and a light chain domain having an amino acid sequence of SEQ ID NO: 5, SEQ ID NO: 6, SEQ ID NO:7, SEQ NO NO: 12, SEQ ID NO: 13, SEQ ID NO: 14 or SEQ ID NO: 15. In some embodiments, the light chain domain has an amino acid sequence of SEQ ID NO: 5. In some embodiments, the light chain domain has an amino acid sequence of SEQ ID NO: 6. In some embodiments, the light chain domain has an amino acid sequence of SEQ ID NO: 7. In some embodiments, the light chain domain has an amino acid sequence of SEQ ID NO: 12. In some embodiments, the light chain domain has an amino acid sequence of SEQ ID NO: 13. In some embodiments, the light chain domain has an amino acid sequence of SEQ ID NO: 14. In some embodiments, the light chain domain has an amino acid sequence of SEQ ID NO: 15.

In some embodiments, compositions comprise a VEGF-A-binding protein comprising a heavy chain domain having an amino acid sequence of SEQ ID NO: 4.

In some embodiments, compositions comprise a VEGF-A-binding protein comprising a light chain domain having an amino acid sequence of SEQ ID NO: 5.

In some embodiments, compositions comprise a VEGF-A-binding protein comprising a light chain domain having an amino acid sequence of SEQ ID NO: 6.

In some embodiments, compositions comprise a VEGF-A-binding protein comprising a light chain domain having an amino acid sequence of SEQ ID NO: 7.

In some embodiments, compositions comprise a VEGF-A-binding protein comprising a light chain domain having an amino acid sequence of SEQ ID NO: 12.

In some embodiments, compositions comprise a VEGF-A-binding protein comprising a light chain domain having an amino acid sequence of SEQ ID NO: 13.

In some embodiments, compositions comprise a VEGF-A-binding protein comprising a light chain domain having an amino acid sequence of SEQ ID NO: 14.

In some embodiments, compositions comprise a VEGF-A-binding protein comprising a light chain domain having an amino acid sequence of SEQ ID NO: 15.

In some embodiments, compositions comprise a HER2-binding protein comprising a heavy chain domain having an amino acid sequence of SEQ ID NO: 21, and a light chain domain having an amino acid sequence of SEQ ID NO: 24 or SEQ ID NO: 25. In some embodiments, the light chain domain has an amino acid sequence of SEQ ID NO: 24. In some embodiments, the light chain domain has an amino acid sequence of SEQ ID NO: 25.

In some embodiments, compositions comprise a HER2-binding protein comprising a heavy chain domain having an amino acid sequence of SEQ ID NO: 23, and a light chain domain having an amino acid sequence of SEQ ID NO: 20.

In some embodiments, compositions comprise a HER2-binding protein comprising a heavy chain domain having an amino acid sequence of SEQ ID NO: 23, and a light chain domain having an amino acid sequence of SEQ ID NO: 24 or SEQ ID NO: 25. In some embodiments, the light chain domain has an amino acid sequence of SEQ ID NO: 24. In some embodiments the light chain domain has an amino acid sequence of SEQ ID NO: 25.

In some embodiments, compositions comprise a HER2-binding protein comprising a heavy chain domain having an amino acid sequence of SEQ ID NO: 23.

In some embodiments, compositions comprise a HER2-binding protein comprising a light chain domain having an amino acid sequence of SEQ ID NO: 24.

In some embodiments, compositions comprise a HER2-binding protein comprising a light chain domain having an amino acid sequence of SEQ ID NO: 25.

In some embodiments, compositions comprise a VEGF-A-binding protein or a HER2-binding protein in the form of an antibody. In some embodiments, compositions comprise a VEGF-A-binding protein in the form of a monoclonal antibody. In some embodiments, the monoclonal antibody is a chimeric monoclonal antibody, human monoclonal antibody or humanized monoclonal antibody.

In some embodiments, compositions comprise a VEGF-A-binding protein or a HER2-binding protein in the form of a fusion protein.

In some embodiments, compositions comprise a VEGF-A-binding protein or a HER2-binding protein in the form of a Fab antibody fragment, single-chain Fv antibody fragment, or minibody.

In some embodiments, compositions comprise a VEGF-A-binding protein or a HER2-binding protein conjugated to a diagnostic agent or a therapeutic agent.

In some embodiments, the compositions comprise the proteins provided herein at a concentration of 20 mg/ml to 350 mg/ml. In some embodiments, the protein concentration may be 30 mg/ml to 340 mg/ml, 40 mg/ml to 330 mg/ml, 50 mg/ml to 320 mg/ml, 60 mg/ml to 310 mg/ml, 70 mg/ml to 300 mg/ml, 80 mg/ml to 290 mg/ml, 90 mg/ml to 280 mg/ml, 100 mg/ml to 270 mg/ml, 110 mg/ml to 260 mg/ml, 120 mg/ml to 250 mg/ml, 130 mg/ml to 240 mg/ml, 140 mg/ml to 230 mg/ml, 150 mg/ml to 220 mg/ml, 160 mg/ml to 210 mg/ml, 170 mg/ml to 200 mg/ml, or 180 mg/ml to 190 mg/ml. In some embodiments, the protein concentration is greater than 100 mg/ml and less than 250 mg/ml. In some embodiments, the protein concentration is 120±20 mg/ml. In some embodiments, the protein concentration is 50 mg/ml, 75 mg/ml, 100 mg/ml, 110 mg/ml, 120 mg/ml, 130 mg/ml, 140 mg/ml, or 150 mg/ml. In some embodiments, the protein concentration is 110 mg/ml, 120 mg/ml, 130 mg/ml, 140 mg/ml, or 150 mg/ml.

In some embodiments, the concentration of the VEGF-A-binding proteins in the compositions is greater than the concentration of bevacizumab (AVASTIN®) formulated for intravenous routes of administration. In some embodiments, the concentration of the HER2-binding proteins in the compositions is greater than the concentration of trastuzumab (HERCEPTIN®) formulated for intravenous routes of administration.

In some embodiments, the compositions provided herein may comprise additional components such as, for example, at least one buffer, at least one stabilizer and/or at least one surfactant.

Examples of buffers that may be used in the compositions provided herein include, without limitation, histidine buffer, acetic acid buffer, citric acid buffer, L-histidine/HCl buffer, and combinations thereof. The buffer(s) may be present at a concentration of 1 mM to 100 mM. In some embodiments, the buffer(s) may be present at a concentration of 1 mM, 10 mM, 20 mM, 30 mM, 40 mM, 50 mM, 60 mM, 70 mM, 80 mM, 90 mM, or 100 mM.

The pH of the compositions may be adjusted to 4.5 to 7.0. In some embodiments, the pH is 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, or 7. In some embodiments, the pH is 5.5. In some embodiments, the pH is 5.5±2. In some embodiments, the pH of the composition is adjusted independently from the buffer used. This pH may be obtained by adjustment with an acid or a base, or by using adequate buffer (or mixtures of buffer), or both.

Examples of stabilizers that may be used, e.g., as a primary stabilizer, in the compositions provided herein include, without limitation, salt (e.g., NaCl), carbohydrate, saccharide, amino acid(s) (e.g., methionine), and sugar (e.g., α,α-trehalose dehydrate). Other examples of stabilizers that may be used, e.g., as a secondary stabilizer, in the compositions provided herein include, without limitation, arginine, ornithine, lysine, histidine, glutamic acid, aspartic acid, isoleucine, leucine, alanine, phenylalanine, tyrosine, tryptophan, methionine, serine or proline (e.g., as a hydrochloride). The stabilizer(s) may be present at a concentration of 5 mM to 500 mM, 5 mM to 25 mM, 15 mM to 250 mM, 100 mM to 500 mM, or 150 mM to 250 mM. In some embodiments, the stabilizer(s) may be present at a concentration of 10 mM, 20 mM, 30 mM, 40 mM, 50 mM, 60 mM, 70 mM, 80 mM, 90 mM, 100 mM, 110 mM, 120 mM, 130 mM, 140 mM, 150 mM, 160 mM, 170 mM, 180 mM, 190 mM, 200 mM, 210 mM, 220 mM, 230 mM, 240 mM, 250 mM, 260 mM, 270 mM, 280 mM, 290 mM, 300 mM, 310 mM, 320 mM, 330 mM, 340 mM, 350 mM, 360 mM, 370 mM, 380 mM, 390 mM, 400 mM, 410 mM, 420 mM, 430 mM, 440 mM, 450 mM, 460 mM, 470 mM, 480 mM, 490 mM, or 500 mM. In some embodiments, the composition may comprise methionine at a concentration of 5 mM to 25 mM, or 5 mM to 15 mM. In some embodiments, the methionine concentration is 10 mM.

Examples of surfactants that may be used in the compositions provided herein include, without limitation, nonionic surfactant(s) such as, for example, polysorbate (e.g., polysorbate 20 and polysorbate 80) and polyethylene-polypropylene copolymer. The surfactant(s) may be present at a concentration of 0.01% to 0.1% (weight/volume, w/v), 0.02% to 0.08% (w/v), 0.02% to 0.06% (w/v), or 0.06% (w/v). In some embodiments, the surfactant(s) may be present at a concentration of 0.02% (w/v), 0.03% (w/v), 0.04% (w/v), 0.05% (w/v), 0.06% (w/v), 0.07% (w/v), 0.08% (w/v), 0.09% (w/v), or 0.1% (w/v).

In some embodiments, VEGF-A-binding proteins or HER2-binding proteins of the compositions provided herein may be lyophilized or in aqueous solution.

In some embodiments, the compositions may or may not be sterile. A sterile composition may be one that is aseptic or free from all living microorganisms and their spores.

In some embodiments, the compositions provided herein may further comprise pharmaceutically acceptable carriers, excipients and/or stabilizers (see, e.g., Remington's Pharmaceutical Sciences 16th edition, Osol, A. Ed., 1980, incorporated herein by reference). As used herein, pharmaceutically acceptable carriers may include without limitation solvents, dispersion media, coatings, antibacterial and antifungal agents, and isotonic and absorption delaying agents. Pharmaceutically acceptable carriers, excipients and/or stabilizers are non-toxic to recipients (e.g., human subjects) at the dosages and concentrations used. In some embodiments, the carriers, excipients and/or stabilizers are suitable for intravenous, intramuscular, subcutaneous, parenteral, spinal or epidermal administration (e.g., by injection or infusion).

Examples of carriers, excipients and/or stabilizers include, without limitation buffers such as phosphate, citrate, and/or other organic acids; antioxidants such as ascorbic acid and/or methionine; preservatives such as octadecyldimethylbenzyl ammonium chloride, hexamethonium chloride, benzalkonium chloride, benzethonium chloride, phenol, butyl or benzyl alcohol, alkyl parabens such as methyl or propyl parabens, catechol, resorcinol, cyclohexanol, 3-pentanol, and/or m-cresol; low molecular weight (e.g., less than 10 residues) polypeptides; proteins such as serum albumin, gelatin, and/or immunoglobulins; hydrophilic polymers such as olyvinylpyrrolidone; amino acids such as glycine, glutamine, asparagine, histidine, arginine, and/or lysine; monosaccharides, disaccharides, and/or other carbohydrates including glucose, mannose, and/or dextrins; chelating agents such as EDTA; sugars such as sucrose, mannitol, trehalose and/or sorbitol; salt-forming counter-ions such as sodium; metal complexes such as Zn-protein complexes; and/or non-ionic surfactants such as TWEEN®, PLURONIC® and/or polyethylene glycol (PEG).

In some embodiments, the compositions may further comprise adjuvants such as, for example, preservatives, wetting agents, emulsifying agents and/or dispersing agents. In some embodiments, the compositions are sterilized and/or may comprise antibacterial and antifungal agents, for example, paraben, chlorobutanol, and/or phenol sorbic acid. In some embodiments, the composition may also comprise isotonic agents, such as sugars and/or sodium chloride. In some embodiments, the composition may further comprise agents that delay absorption such as aluminum monostearate and/or gelatin.

In some embodiments, the compositions provided herein further comprise at least one additional active agent including, without limitation, cytotoxic agents, chemotherapeutic agents, cytokines and/or immunosuppressive agents.

In some embodiments, a composition provided herein is substantially free of any additive that reduces aggregation of VEGF-A-binding proteins or HER2-binding proteins.

In some embodiments, the compositions provided herein comprise VEGF-A-binding proteins or HER2-binding proteins entrapped in microcapsules prepared, for example, by coacervation techniques (e.g., hydroxymethylcellulose or gelatin-microcapsules) or by interfacial polymerization (e.g., poly-(methylmethacylate) microcapsules). In some embodiments, the compositions provided herein comprise VEGF-A-binding proteins or HER2-binding proteins entrapped in colloidal drug delivery systems such as, for example, liposomes, albumin microspheres, microemulsions, nano-particles and nanocapsules. In some embodiments, the compositions provided herein comprise VEGF-A-binding proteins or HER2-binding proteins entrapped in macroemulsions. See, e.g., Remington's Pharmaceutical Sciences 16th edition, Osol, A. Ed. (1980).

In some embodiments, the compositions provided herein are sustained-release compositions.

Some embodiments provide highly concentrated, stable liquid compositions of

VEGF-A-binding proteins or HER2-binding proteins having a reduced tendency to aggregate. As used herein, “highly concentrated” compositions may refer to compositions comprising the proteins provided herein at a concentration of greater than 100 mg/ml (e.g., 110 mg/ml to 150 mg/ml). As used herein, “stable” compositions may refer to compositions comprising the proteins provided herein that retain (or essentially retain) physical stability and/or chemical stability and/or biological activity upon storage for an intended period of time (e.g., intended shelf-life of the composition) at an intended temperature (e.g., 2-8° C.). In some embodiments, the compositions are stable following freezing (to, e.g., −20° C.) and thawing of the compositions, for example, following one or more cycles of freezing and thawing. Various analytical techniques for measuring protein stability are available in the art (see, e.g., Peptide and Protein Drug Delivery, 247-301, Vincent Lee Ed., Marcel Dekker, Inc., New York, N.Y., Pubs., 1991; and Jones, A. Adv. Drug Delivery Rev., 1993, 10: 29-90, each of which are incorporated herein by reference). Stability may be measured at a select temperature for a select time period. Stability may be evaluated qualitatively and/or quantitatively in a variety of different ways including, without limitation, evaluation of aggregate formation (e.g., using size exclusion chromatography, measuring turbidity, and/or visual inspection); assessment of charge heterogeneity using cation exchange chromatography or capillary zone electrophoresis; SDS-PAGE analysis to compare reduced and intact antibody; and/or evaluating biological activity or antigen binding function of the protein. Instability may involve aggregation, deamidation (e.g. Asn deamidation), oxidation (e.g., Met oxidation), isomerization (e.g. Asp isomeriation), clipping/hydrolysis/fragmentation (e.g.,. hinge region fragmentation), succinimide formation, and/or unpaired cysteine(s).

In some embodiments, the compositions, for example, the highly concentrated compositions, are formulated for subcutaneous or intramuscular delivery. In some embodiments, the compositions provided herein are considered to be highly concentrated compositions formulated for subcutaneous delivery if the protein concentration in the compositions is greater than 100 mg/ml (e.g., 110 mg/ml to 150 mg/ml) and the volume of the compositions is 2 ml or is less than 2 ml. In some embodiments, the volume of the compositions is 1.5 ml. In some embodiments, the volume of the compositions is 0.5 ml to 3.5 ml, 1.0 ml to 3.0 ml, 1.5 ml to 2.5 ml, or 1.5 ml to 2.0 ml.

Examples of highly concentrated compositions comprising VEGF-A-binding proteins or HER2-binding proteins include, without limitation, the following:

(a) 100 to 150 mg/ml protein, 1 to 50 mM of a histidine buffer (e.g., L-histidine/HCl) at a pH of 5.5, 15 to 250 mM of a stabilizer (e.g., α,α-trehalose dihydrate) and, optionally, methionine as a second stabilizer at a concentration of 5 to 25 mM, and polysorbate 20 or polysorbate 80 at a concentration of 0.02 to 0.06% (w/v);

(b) 120±20 mg/ml protein, 10 to 30 mM or 20 mM of a histidine buffer (e.g., L-histidine/HCl) at a pH of 5.5, 150 to 250 mM or 210 mM of a stabilizer (e.g., α,α-trehalose dihydrate) and, optionally, methionine as a second stabilizer at a concentration of 5 to 25 mM, 5 to 15 mM or 10 mM, and polysorbate 20 or polysorbate 80 at a concentration of 0.02 to 0.06% (w/v);

(c) 120 mg/ml protein, 10 to 30 mM or 20 mM of a histidine buffer (e.g., L-histidine/HCl) at a pH of 5.5, 150 to 250 mM or 210 mM of a stabilizer (e.g., α,α-trehalose dihydrate) and, optionally, methionine as a second stabilizer at a concentration of 5 to 25 mM, 5 to 15 mM, or 10 mM, and polysorbate 20 or polysorbate 80 at a concentration of 0.02 to 0.06% (w/v); and

(d) 120 mg/ml protein, 20 mM of L-histidine/HCl at a pH of 5.5, 210 mM α,α-trehalose dihydrate and, optionally, 10 mM methionine as a second stabilizer, and polysorbate 20 or polysorbate 80 at a concentration of 0.02 to 0.06% (w/v).

### Methods of Use

Some embodiments provide methods of using VEGF-A-binding proteins or HER2-binding proteins and composition, for example, in in vitro, in situ and in vivo applications.

Examples of in vitro and in situ applications in accordance with the present disclosure include, without limitation, cell killing assays, as positive controls for apoptosis assays, for purification or immunoprecipitation of antigen from cells, and for diagnostic assays.

Examples of in vivo applications in accordance with the present disclosure include, without limitation, methods of treatment. In some embodiments, the proteins and protein compositions may be used to treat one or more condition(s) such as, for example, cancers and/or non-malignant conditions in a subject in need thereof. Thus, in some embodiments, provided herein are methods of treating cancers and/or non-malignant conditions in a subject in need thereof. In some embodiments, the methods of treatment comprise administering to the subject in need thereof a therapeutically effective amount of the proteins and/or compositions. The term “therapeutically effective amount” is described below.

In some embodiments, the proteins provided herein are administered at dosages of 0.0001 mg/kg to 100 mg/kg of the recipient (e.g., subject) body weight. In some embodiments, the proteins are administered at dosages of 0.01 mg/kg to 5 mg/kg of the recipient body weight. In some embodiments, the proteins are administered at dosages of 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1.0 mg/kg, 1.1 mg/kg, 1.2 mg/kg, 1.3 mg/kg, 1.4 mg/kg, 1.5 mg/kg, 1.6 mg/kg, 1.7 mg/kg, 1.8 mg/kg, 1.9 mg/kg, 2.0 mg/kg, 2.1 mg/kg, 2.2 mg/kg, 2.3 mg/kg, 2.4 mg/kg, 2.5 mg/kg, 2.6 mg/kg, 2.7 mg/kg, 2.8 mg/kg, 2.9 mg/kg, 3.0 mg/kg, 3.1 mg/kg, 3.2 mg/kg, 3.3 mg/kg, 3.4 mg/kg, 3.5 mg/kg, 3.6 mg/kg, 3.7 mg/kg, 3.8 mg/kg, 3.9 mg/kg, 4.0 mg/kg, 4.1 mg/kg, 4.2 mg/kg, 4.3 mg/kg, 4.4 mg/kg, 4.5 mg/kg, 4.6 mg/kg, 4.7 mg/kg, 4.8 mg/kg, 4.9 mg/kg, 5.0 mg/kg, 5.1 mg/kg, 5.2 mg/kg, 5.3 mg/kg, 5.4 mg/kg, 5.5 mg/kg, 5.6 mg/kg, 5.7 mg/kg, 5.8 mg/kg, 5.9 mg/kg, 6.0 mg/kg, 6.1 mg/kg, 6.2 mg/kg, 6.3 mg/kg, 6.4 mg/kg, 6.5 mg/kg, 6.6 mg/kg, 6.7 mg/kg, 6.8 mg/kg, 6.9 mg/kg, 7.0 mg.kg, 7.1 mg/kg, 7.2 mg/kg, 7.3 mg/kg, 7.4 mg/kg, 7.5 mg/kg, 7.6 mg/kg, 7.7 mg/kg, 7.8 mg/kg, 7.9 mg/kg, 8.0 mg/kg, 8.1 mg/kg, 8.2 mg/kg, 8.3 mg/kg, 8.4 mg/kg, 8.5 mg/kg, 8.6 mg/kg, 8.7 mg/kg, 8.8 mg/kg, 8.9 mg/kg, 9.0 mg/kg, 9.1 mg/kg, 9.2 mg/kg, 9.3 mg/kg, 9.4 mg/kg, 9.5 mg/kg, 9.6 mg/kg, 9.7 mg/kg, 9.8 mg/kg, 9.9 mg/kg, or 10 mg/ml recipient body weight. In some embodiments, the proteins are administered at dosages of 0.3 mg/kg body weight, 1 mg/kg recipient body weight, 3 mg/kg recipient body weight, 5 mg/kg recipient body weight, 10 mg/kg recipient body weight, or within the range of 1-10 mg/kg recipient body weight.

In some embodiments, the proteins provided herein are administered once per week, once every two weeks, once every three weeks, once every four weeks, once a month, once every three months or once every three to six months.

Dosage and frequency may vary depending on the half-life of the protein in the recipient. For example, in general, human antibodies show the longest half-life, followed by humanized antibodies, chimeric antibodies, and nonhuman antibodies.

Actual dosage levels of the proteins provided herein may be varied so as to obtain an amount of the proteins which is effective to achieve the desired response (e.g., therapeutic response) for a particular subject, composition, and mode of administration, without being toxic to the subject. The selected dosage level may depend upon a variety of pharmacokinetic factors including, without limitation, the activity of the proteins, the route of administration, the time of administration, the rate of excretion of the proteins, the duration of administration, other drugs, compounds and/or materials used in combination with the proteins, the age, sex, weight, condition, general health and prior medical history of the subject.

In some embodiments, the proteins and protein compositions provided herein may be administered at therapeutically effective amounts. As used herein, “therapeutically effective amount” of the proteins and/or the protein compositions may result in a decrease in severity of the symptoms of a condition, an increase in frequency and duration of symptom-free periods, or a prevention of impairment or disability due to the condition. For example, for administration of the proteins or compositions to a subject having tumors, a “therapeutically effective amount” inhibits cell growth or tumor growth by at least 20%, by at least 40%, by at least 60%, or by at least 80% relative to untreated subjects. The ability of the proteins to inhibit tumor growth may be evaluated in an animal model system predictive of efficacy in human tumors. Alternatively, the proteins may be evaluated by examining the ability of the proteins to inhibit tumor growth in vitro. In some embodiments, a therapeutically effective amount of the VEGF-A-binding proteins or HER2-binding proteins may decrease tumor size, or otherwise ameliorate symptoms in a subject. One of ordinary skill in the art would be able to determine such amounts based on such factors as the subject's size, the severity of the subject's symptoms, and the particular composition or route of administration selected.

In some embodiments, the proteins and/or the compositions provided herein may be administered via one or more routes of administration using one or more of a variety of methods known in the art. In some embodiments, the proteins and/or the protein compositions are administered via subcutaneous, intravenous, intramuscular, intradermal, intraperitoneal, spinal or other parenteral routes of administration, for example by injection or infusion. As used herein, the term “parenteral administration” may refer to modes of administration other than enteral and topical administration, usually by injection, and includes, without limitation, subcutaneous, intravenous, intramuscular, intraarterial, intrathecal, intracapsular, intraorbital, intracardiac, intradermal, intraperitoneal, transtracheal, subcuticular, intraarticular, subcapsular, subarachnoid, intraspinal, epidural and intrasternal injection, and infusion. In other embodiments, the proteins and/or the protein compositions may be administered via a nonparenteral route, such as a topical, epidermal or mucosal route of administration, for example, intranasally, orally, vaginally, rectally, sublingually or topically.

In some embodiments, the VEGF-A-binding proteins, HER2-binding proteins and compositions comprising VEGF-A-binding proteins or HER2-binding proteins are used to treat cancers such as, for example, colorectal cancer, lung cancer, breast cancer, glioblastoma, kidney cancer, ovarian cancer, and/or other cancers expressing or overexpressing VEGF-A or HER2.

### Kits

Some embodiments provide kits comprising VEGF-A-binding proteins or HER2-binding proteins and compositions described herein. In some embodiments, kits may comprise a single container (e.g., a syringe or vial) containing a protein or composition. In some embodiment, the volume of the composition may be less than or equal to 2 ml. In such embodiments, the concentration of the protein is greater than 100 mg/ml, for example 110 mg/ml, 120 mg/ml, 130 mg/ml, 140 mg/ml, or 150 mg/ml. The kits may comprise a container(s) and a label or package insert on or associated with the container. Suitable containers include without limitation bottles, vials, and syringes. The containers may be formed from a variety of materials such as glass or plastic. The containers may hold a composition which may be administered to a subject and may have a sterile access port (for example the container may be an intravenous solution bag or a vial having a stopper pierceable by a hypodermic injection needle).

The label or package insert may indicate that kits are used for administering the proteins or protein compositions to a subject. The label or package insert may further comprise instructions for administration. As used herein, a “package insert” may refer to instructions customarily included in commercial packages of products that contain information about the product and its use, for example, indications, dosage, administration, contraindications and/or warnings concerning the use of such products. In some embodiments, the package insert may indicate that the proteins and protein compositions are used for treating cancer or autoimmune conditions.

Additionally, the kits may further comprise other containers comprising additional components including, without limitation, pharmaceutically-acceptable buffers, bacteriostatic water for injection (BWFI), phosphate-buffered saline, Ringer's solution and/or dextrose solution. The kits may also comprise other materials including, without limitation, other buffers, diluents, filters, needles, and/or syringes.

In some embodiments, provided herein are kits that may be useful for various purposes, e.g., for cell killing assays, as a positive control for apoptosis assays, for purification or immunoprecipitation of antigen from cells. For isolation and purification of antigen, the kit may contain an antibody coupled to beads (e.g., sepharose beads). Kits may contain the antibodies for detection and quantitation of VEGF-A or HER2 in vitro, e.g., in an ELISA or a Western blot.

### Examples

### Stabilizing Hyperglycosylated Variants

Increasing stability of VEGF-A-binding proteins was achieved by introducing glycosylation sites near aggregation-prone regions of the protein. To reduce the aggregation propensity of bevacizumab, we intended to mask residues V110, L154, L180 and L201 with a glycosylation motif. Each of these residues, upon mutation to hydrophilic and/or charged residues, increases the stability of bevacizumab.

To generate candidate N-glycosylation sites, i.e., NXS or NXT (X is any amino acid but P), close enough for the sugar group to mask those residues of interest, all of the serine, threonine and asparagine within 10 Å of aggregation-prone regions within the constant domains of the Fab region of bevacizumab (CH1 and CL domains) were identified. The neighboring residues (residues i−2 and i+2) were then considered as candidates for mutation to generate a foreseeable glycosylation site. Eight residues were identified for site-directed mutagenesis to create potential glycosylation sites.

### Glycoengineered Proteins for Increased Stability Against Aggregation

Four variants L₁₁₈N, E₁₉₅N, Q₁₆₀N and Q₁₆₀S were produced in HEK293 human embryonic kidney cells, which are able to carry out the original post-translational modifications and should produce mAbs with glycosylation at the engineered sites. To test whether the introduction of an N-glycosylation site near an aggregation-prone region led to an overall reduction in the aggregation propensity of bevacizumab, the hyperglycosylated variants were expressed, purified and characterized by DSC, turbidity evaluation and SEC-HPLC. The incorporation of N-glycan on the Fab domain of our bevacizumab variants was verified by reducing SDS-PAGE. The heavy chain (HC) of L₁₁₈N and the light chain (LC) of E₁₉₅N and Q₁₆₀N/S have higher molecular weights than those of the wild type (WT), corresponding to the addition of an N-glycan (data not shown). All variants were tested by differential scanning calorimetry (DSC) for their thermal stability. Three transition temperatures were extracted from the obtained thermograms. The introduction of the N-glycan motif on the Fab domain of bevacizumab does not affect the transition temperature of the CH3 domain (Tm3), which changed by less than 0.7° C. The Fab domain shows a higher thermal stability when hyperglycosylated at one of the four positions we tested (Tm2 increased by 1.6° C. to 2.3° C.). Surprisingly, the transition temperature Tm1 attributed to the CH2 domain is also affected. Glycosylation of the sites N₁₉₅, N₁₆₀ and N₁₅₈ increased the CH2 domain stability by 1.6° C. to 2.1° C., whereas glycosylation of N₁₁₈ reduced the CH2 domain thermal stability by 2.3° C.

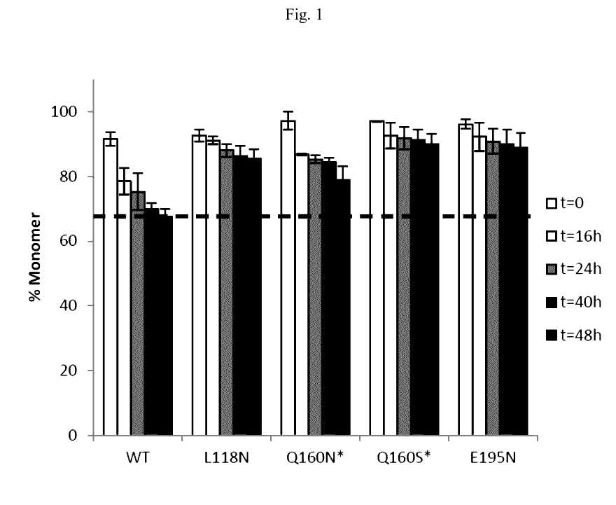

Hyperglycosylated variants were tested for their stability through accelerated aggregation studies at an elevated temperature. 52° C. was chosen as the temperature to accelerate aggregation, and monomer concentrations were monitored over a 48-h period by SEC-HPLC (FIG. 1). The SEC-HPLC data are the measure of the monomer concentration at various time points, and FIG. 1 represents the average of three independent experiments (i.e., three different protein production batches) unless stated otherwise. The presence of soluble aggregates was observed in all samples from the beginning of the experiment, which was due to the high concentration formulation (50 mg/mL) that was close to the solubility limit of bevacizumab. The wild-type bevacizumab sample contained 8.5% soluble aggregates at t=0, whereas the variants Q₁₆₀N, Q₁₆₀S and E₁₉₅N contained less than 4% soluble aggregates at t=0. After 48 h incubation at 52° C., 32% soluble aggregates were observed for the WT, whereas another variant displayed less than 10% aggregates, representing over a 3-fold increase in stabilization. The four hyperglycosylated variants are all more stable than the WT bevacizumab against heat-induced aggregation, having a 1.5 to 3.2-fold increase in stability. A second-order rate constant was extracted from the fitting of the monomer loss measured over time for each variant. The WT and the Q₁₆₀N variant display a fast loss of monomer in the first 16 h of incubation at 52° C., while the variants L₁₁₈N, Q₁₆₀S and E₁₉₅N stayed relatively stable during this incubation period. They were also the three variants with the most stabilizing effect, and their aggregation rate was reduced by a factor 3.3 to 7.4 (FIG. 1). Using SEC-HPLC data and by comparing the amount of monomer measured at each time point (t=16 h, t=24 h, t=48 h) to the amount of monomer at t=0, the amount of insoluble aggregates that did not enter the separation column were estimated. Hyperglycosylated variants, Q₁₆₀N/S and E₁₉₅N exhibited reduced insoluble aggregates (3-5%) compared to the WT (˜8%), whereas L₁₁₈N exhibited nearly double the amount of insoluble aggregates (15%), clearly making the variant L₁₁₈N the least effective hyperglycosylated variant overall (1.3-fold stabilization), although still less aggregation-prone than unmodified bevacizumab. This result was confirmed by turbidity measurements: Abs320 nm=0.014 to 0.052 for the variants, Abs320 nm=0.216 for L118N versus Abs320 nm=0.091 for WT.

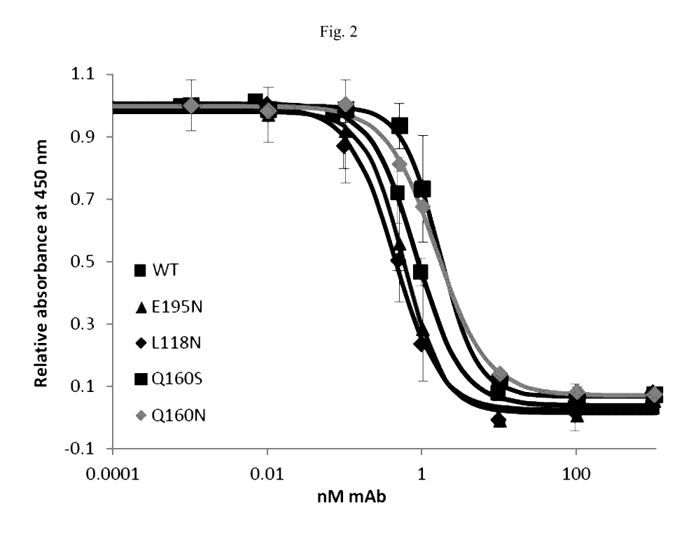

The binding efficacy of the hyperglycosylated engineered bevacizumab was estimated in vitro via a competitive ELISA, allowing measurement of the affinity of the WT and variants for the antigen, VEGF. FIG. 2 shows the binding curve of WT bevacizumab and the hyperglycosylated variants to VEGF-A. The mAbs were preincubated with VEGF, which were then incubated with anti-VEGF IgG1 for further detection by ELISA. A low absorbance at 450 nm indicates a low amount of VEGF bound to the ELISA plate and a high amount of the mAb bound to VEGF. These competitive assay results show that all of the mutations introduced in the Fab fragment, far from the CDR, produce hyperglycosylated variants that bind to VEGF with the same affinity as the commercial drug, the WT bevacizumab and the variants described above. The results indicate that, when glycosylation site locations are chosen carefully, N-glycans can stabilize mAbs without compromising their activity in vitro.

### SEQUENCES

Amino acid sequence of a light chain domain of bevacizumab
SEQ ID NO: 1
DIQMTQSPSS LSASVGDRVT ITCSASQDIS NYLNWYQQKP GKAPKVLIYF TSSLHSGVPS
RFSGSGSGTD FTLTISSLQP EDFATYYCQQ YSTVPWTFGQ GTKVEIKRTV AAPSVFIFPP
SDEQLKSGTA SVVCLLNNFY PREAKVQWKV DNALQSGNSQ ESVTEQDSKD STYSLSSTLT
LSKADYEKHK VYACEVTHQG LSSPVTKSFN RGE
Amino acid sequence of a Fab heavy chain domain of bevacizumab
SEQ ID NO: 2
EVQLVESGGG LVQPGGSLRL SCAASGYTFT NYGMNWVRQA PGKGLEWVGW INTYTGEPTY
AADFKRRFTF SLDTSKSTAY LQMNSLRAED TAVYYCAKYP HYYGSSHWYF DVWGQGTLVT
VSSASTKGPS VFPLAPSSKS TSGGTAALGC LVKDYFPEPV TVSWNSGALT SGVHTFPAVL
QSSGLYSLSS VVTVPSSSLG TQTYICNVNH KPSNTKVDKK VEPK
Amino acid sequence of an Fc domain of bevacizumab
SEQ ID NO: 3
SCDKTHTCPP CPAPELLGGP SVFLFPPKPK DTLMISRTPE VTCVVVDVSH EDPEVKFNWY
VDGVEVHNAK TKPREEQYNS TYRVVSVLTV LHQDWLNGKE YKCKVSNKAL PAPIEKTISK
AKGQPREPQV YTLPPSRDEL TKNQVSLTCL VKGFYPSDIA VEWESNGQPE NNYKTTPPVL
DSDGSFFLYS KLTVDKSRWQ QGNVFSCSVM HEALHNHYTQ KSLSLSPGK
Amino acid sequence of a Fab heavy chain domain of bevacizumab
modified at position L₁₁₈N.
SEQ ID NO: 4
EVQLVESGGG LVQPGGSLRL SCAASGYTFT NYGMNWVRQA PGKGLEWVGW INTYTGEPTY
AADFKRRFTF SLDTSKSTAY LQMNSLRAED TAVYYCAKYP HYYGSSHWYF DVWGQGTNVT
VSSASTKGPS VFPLAPSSKS TSGGTAALGC LVKDYFPEPV TVSWNSGALT SGVHTFPAVL
QSSGLYSLSS VVTVPSSSLG TQTYICNVNH KPSNTKVDKK VEPK
Amino acid sequence of a light chain domain of bevacizumab
modified at position E₁₉₅N.
SEQ ID NO: 5
DIQMTQSPSS LSASVGDRVT ITCSASQDIS NYLNWYQQKP GKAPKVLIYF TSSLHSGVPS
RFSGSGSGTD FTLTISSLQP EDFATYYCQQ YSTVPWTFGQ GTKVEIKRTV AAPSVFIFPP
SDEQLKSGTA SVVCLLNNFY PREAKVQWKV DNALQSGNSQ ESVTEQDSKD STYSLSSTLT
LSKADYEKHK VYACNVTHQG LSSPVTKSFN RGE
Amino acid sequence of a light chain domain of bevacizumab
modified at position Q₁₆₀N.
SEQ ID NO: 6
DIQMTQSPSS LSASVGDRVT ITCSASQDIS NYLNWYQQKP GKAPKVLIYF TSSLHSGVPS
RFSGSGSGTD FTLTISSLQP EDFATYYCQQ YSTVPWTFGQ GTKVEIKRTV AAPSVFIFPP
SDEQLKSGTA SVVCLLNNFY PREAKVQWKV DNALQSGNSN ESVTEQDSKD STYSLSSTLT
LSKADYEKHK VYACEVTHQG LSSPVTKSFN RGE
Amino acid sequence of a light chain domain of bevacizumab
modified at position Q₁₆₀S.
SEQ ID NO: 7
DIQMTQSPSS LSASVGDRVT ITCSASQDIS NYLNWYQQKP GKAPKVLIYF TSSLHSGVPS
RFSGSGSGTD FTLTISSLQP EDFATYYCQQ YSTVPWTFGQ GTKVEIKRTV AAPSVFIFPP
SDEQLKSGTA SVVCLLNNFY PREAKVQWKV DNALQSGNSS ESVTEQDSKD STYSLSSTLT
LSKADYEKHK VYACEVTHQG LSSPVTKSFN RGE
Amino acid sequence of Vascular Endothelial Growth Factor A
(VEGF-A) (GenBank Protein ID AAA35789.1)
SEQ ID NO: 8
MNFLLSWVHWSLALLLYLHHAKWSQAAPMAEGGGQNHHEVVKFMDVYQRSYCHPIETLVDIFQEYPDEIEYIFKP
SCVPLMRCGGCCNDEGLECVPTEESNITMQIMRIKPHQGQHIGEMSFLQHNKCECRPKKDRARQEKKSVRGKGKG
QKRKRKKSRYKSWSVYVGARCCLMPWSLPGPHPCGPCSERRKHLFVQDPQTCKCSCKNTDSRCKARQLELNERTC
RCDKPRR
Amino acid sequence of a Fab heavy chain domain of bevacizumab
modified at position L₁₁₈
SEQ ID NO: 9
EVQLVESGGG LVQPGGSLRL SCAASGYTFT NYGMNWVRQA PGKGLEWVGW INTYTGEPTY
AADFKRRFTF SLDTSKSTAY LQMNSLRAED TAVYYCAKYP HYYGSSHWYF DVWGQGTXVT
VSSASTKGPS VFPLAPSSKS TSGGTAALGC LVKDYFPEPV TVSWNSGALT SGVHTFPAVL
QSSGLYSLSS VVTVPSSSLG TQTYICNVNH KPSNTKVDKK VEPK
Amino acid sequence of a Fab heavy chain domain of bevacizumab
modified at position E₁₉₅
SEQ ID NO: 10
DIQMTQSPSS LSASVGDRVT ITCSASQDIS NYLNWYQQKP GKAPKVLIYF TSSLHSGVPS
RFSGSGSGTD FTLTISSLQP EDFATYYCQQ YSTVPWTFGQ GTKVEIKRTV AAPSVFIFPP
SDEQLKSGTA SVVCLLNNFY PREAKVQWKV DNALQSGNSQ ESVTEQDSKD STYSLSSTLT
LSKADYEKHK VYACXVTHQG LSSPVTKSFN RGE
Amino acid sequence of a Fab heavy chain domain of bevacizumab
modified at position Q₁₆₀
SEQ ID NO: 11
DIQMTQSPSS LSASVGDRVT ITCSASQDIS NYLNWYQQKP GKAPKVLIYF TSSLHSGVPS
RFSGSGSGTD FTLTISSLQP EDFATYYCQQ YSTVPWTFGQ GTKVEIKRTV AAPSVFIFPP
SDEQLKSGTA SVVCLLNNFY PREAKVQWKV DNALQSGNSX ESVTEQDSKD STYSLSSTLT
LSKADYEKHK VYACEVTHQG LSSPVTKSFN RGE
Amino acid sequence of a light chain domain of bevacizumab
modified at position K₁₀₇N
SEQ ID NO: 12
DIQMTQSPSS LSASVGDRVT ITCSASQDIS NYLNWYQQKP GKAPKVLIYF TSSLHSGVPS
RFSGSGSGTD FTLTISSLQP EDFATYYCQQ YSTVPWTFGQ GTKVEINRTV AAPSVFIFPP
SDEQLKSGTA SVVCLLNNFY PREAKVQWKV DNALQSGNSS ESVTEQDSKD STYSLSSTLT
LSKADYEKHK VYACEVTHQG LSSPVTKSFN RGE
Amino acid sequence of a light chain domain of bevacizumab
modified at position Y₁₄₀S
SEQ ID NO: 13
DIQMTQSPSS LSASVGDRVT ITCSASQDIS NYLNWYQQKP GKAPKVLIYF TSSLHSGVPS
RFSGSGSGTD FTLTISSLQP EDFATYYCQQ YSTVPWTFGQ GTKVEIKRTV AAPSVFIFPP
SDEQLKSGTA SVVCLLNNFS PREAKVQWKV DNALQSGNSS ESVTEQDSKD STYSLSSTLT
LSKADYEKHK VYACEVTHQG LSSPVTKSFN RGE
Amino acid sequence of a light chain domain of bevacizumab
modified at position K₁₆₉N
SEQ ID NO: 14
DIQMTQSPSS LSASVGDRVT ITCSASQDIS NYLNWYQQKP GKAPKVLIYF TSSLHSGVPS
RFSGSGSGTD FTLTISSLQP EDFATYYCQQ YSTVPWTFGQ GTKVEIKRTV AAPSVFIFPP
SDEQLKSGTA SVVCLLNNFY PREAKVQWKV DNALQSGNSS ESVTEQDSND STYSLSSTLT
LSKADYEKHK VYACEVTHQG LSSPVTKSFN RGE
Amino acid sequence of a light chain domain of bevacizumab
modified at position D₁₇₀N
SEQ ID NO: 15
DIQMTQSPSS LSASVGDRVT ITCSASQDIS NYLNWYQQKP GKAPKVLIYF TSSLHSGVPS
RFSGSGSGTD FTLTISSLQP EDFATYYCQQ YSTVPWTFGQ GTKVEIKRTV AAPSVFIFPP
SDEQLKSGTA SVVCLLNNFY PREAKVQWKV DNALQSGNSS ESVTEQDSKN STYSLSSTLT
LSKADYEKHK VYACEVTHQG LSSPVTKSFN RGE
Amino acid sequence of a light chain domain of bevacizumab
modified at position K₁₀₇
SEQ ID NO: 16
DIQMTQSPSS LSASVGDRVT ITCSASQDIS NYLNWYQQKP GKAPKVLIYF TSSLHSGVPS
RFSGSGSGTD FTLTISSLQP EDFATYYCQQ YSTVPWTFGQ GTKVEIXRTV AAPSVFIFPP
SDEQLKSGTA SVVCLLNNFY PREAKVQWKV DNALQSGNSS ESVTEQDSKD STYSLSSTLT
LSKADYEKHK VYACEVTHQG LSSPVTKSFN RGE
Amino acid sequence of a light chain domain of bevacizumab
modified at position Y₁₄₀
SEQ ID NO: 17
DIQMTQSPSS LSASVGDRVT ITCSASQDIS NYLNWYQQKP GKAPKVLIYF TSSLHSGVPS
RFSGSGSGTD FTLTISSLQP EDFATYYCQQ YSTVPWTFGQ GTKVEIKRTV AAPSVFIFPP
SDEQLKSGTA SVVCLLNNFX PREAKVQWKV DNALQSGNSS ESVTEQDSKD STYSLSSTLT
LSKADYEKHK VYACEVTHQG LSSPVTKSFN RGE
Amino acid sequence of a light chain domain of bevacizumab
modified at position K₁₆₉
SEQ ID NO: 18
DIQMTQSPSS LSASVGDRVT ITCSASQDIS NYLNWYQQKP GKAPKVLIYF TSSLHSGVPS
RFSGSGSGTD FTLTISSLQP EDFATYYCQQ YSTVPWTFGQ GTKVEIKRTV AAPSVFIFPP
SDEQLKSGTA SVVCLLNNFY PREAKVQWKV DNALQSGNSS ESVTEQDSXD STYSLSSTLT
LSKADYEKHK VYACEVTHQG LSSPVTKSFN RGE
Amino acid sequence of a light chain domain of bevacizumab
modified at position D₁₇₀
SEQ ID NO: 19
DIQMTQSPSS LSASVGDRVT ITCSASQDIS NYLNWYQQKP GKAPKVLIYF TSSLHSGVPS
RFSGSGSGTD FTLTISSLQP EDFATYYCQQ YSTVPWTFGQ GTKVEIKRTV AAPSVFIFPP
SDEQLKSGTA SVVCLLNNFY PREAKVQWKV DNALQSGNSS ESVTEQDSKX STYSLSSTLT
LSKADYEKHK VYACEVTHQG LSSPVTKSFN RGE
Amino acid sequence of a light chain domain of trastuzumab
SEQ ID NO: 20
DIQMTQSPSS LSASVGDRVT ITCRASQDVN TAVAWYQQKP GKAPKLLIYS ASFLYSGVPS
RFSGSRSGTD FTLTISSLQP EDFATYYCQQ HYTTPPTFGQ GTKVEIKRTV AAPSVFIFPP
SDEQLKSGTA SVVCLLNNFY PREAKVQWKV DNALQSGNSQ ESVTEQDSKD STYSLSSTLT
LSKADYEKHK VYACEVTHQG LSSPVTKSFN RGEC
Amino acid sequence of a Fab heavy chain domain of trastuzumab
SEQ ID NO: 21
EVQLVESGGG LVQPGGSLRL SCAASGFNIK DTYIHWVRQA PGKGLEWVAR IYPTNGYTRY
ADSVKGRFTI SADTSKNTAY LQMNSLRAED TAVYYCSRWG GDGFYAMDYW GQGTLVTVSS
ASTKGPSVFP LAPSSKSTSG GTAALGCLVK DYFPEPVTVS WNSGALTSGV HTFPAVLQSS
GLYSLSSVVT VPSSSLGTQT YICNVNHKPS NTKVDKKVEP
Amino acid sequence of an Fc domain of trastuzumab
SEQ ID NO: 22
PKSCDKTHTC PPCPAPELLG GPSVFLFPPK PKDTLMISRT PEVTCVVVDV SHEDPEVKFN
WYVDGVEVHN AKTKPREEQY NSTYRVVSVL TVLHQDWLNG KEYKCKVSNK ALPAPIEKTI
SKAKGQPREP QVYTLPPSRD ELTKNQVSLT CLVKGFYPSD IAVEWESNGQ PENNYKTTPP
VLDSDGSFFL YSKLTVDKSR WQQGNVFSCS VMHEALHNHY TQKSLSLSPG K
Amino acid sequence of a Fab heavy chain domain of trastuzumab
modified at position L₁₁₅N.
SEQ ID NO: 23
EVQLVESGGG LVQPGGSLRL SCAASGFNIK DTYIHWVRQA PGKGLEWVAR IYPTNGYTRY
ADSVKGRFTI SADTSKNTAY LQMNSLRAED TAVYYCSRWG GDGFYAMDYW GQGTNVTVSS
ASTKGPSVFP LAPSSKSTSG GTAALGCLVK DYFPEPVTVS WNSGALTSGV HTFPAVLQSS
GLYSLSSVVT VPSSSLGTQT YICNVNHKPS NTKVDKKVEP
Amino acid sequence of a light chain domain of trastuzumab
modified at position Q₁₆₀N.
SEQ ID NO: 24
DIQMTQSPSS LSASVGDRVT ITCRASQDVN TAVAWYQQKP GKAPKLLIYS ASFLYSGVPS
RFSGSRSGTD FTLTISSLQP EDFATYYCQQ HYTTPPTFGQ GTKVEIKRTV AAPSVFIFPP
SDEQLKSGTA SVVCLLNNFY PREAKVQWKV DNALQSGNSN ESVTEQDSKD STYSLSSTLT
LSKADYEKHK VYACEVTHQG LSSPVTKSFN RGEC
Amino acid sequence of a light chain domain of trastuzumab
modified at position E₁₉₅N.
SEQ ID NO: 25
DIQMTQSPSS LSASVGDRVT ITCRASQDVN TAVAWYQQKP GKAPKLLIYS ASFLYSGVPS
RFSGSRSGTD FTLTISSLQP EDFATYYCQQ HYTTPPTFGQ GTKVEIKRTV AAPSVFIFPP
SDEQLKSGTA SVVCLLNNFY PREAKVQWKV DNALQSGNSQ ESVTEQDSKD STYSLSSTLT
LSKADYEKHK VYACNVTHQG LSSPVTKSFN RGEC
Amino acid sequence of Human Epidermal Growth Factor Receptor 2
(HER2; also refered to as receptor tyrosine-protein kinase
erbB-2 precursor) (NCBI Reference Sequence NP_004439).
SEQ ID NO: 26
MELAALCRWGLLLALLPPGAASTQVCTGTDMKLRLPASPETHLDMLRHLYQGCQVVQGNLELTYLPTNASLSFLQ
DIQEVQGYVLIAHNQVRQVPLQRLRIVRGTQLFEDNYALAVLDNGDPLNNTTPVTGASPGGLRELQLRSLTEILK
GGVLIQRNPQLCYQDTILWKDIFHKNNQLALTLIDTNRSRACHPCSPMCKGSRCWGESSEDCQSLTRTVCAGGCA
RCKGPLPTDCCHEQCAAGCTGPKHSDCLACLHFNHSGICELHCPALVTYNTDTFESMPNPEGRYTFGASCVTACP
YNYLSTDVGSCTLVCPLHNQEVTAEDGTQRCEKCSKPCARVCYGLGMEHLREVRAVTSANIQEFAGCKKIFGSLA
FLPESFDGDPASNTAPLQPEQLQVFETLEEITGYLYISAWPDSLPDLSVFQNLQVIRGRILHNGAYSLTLQGLGI
SWLGLRSLRELGSGLALIHHNTHLCFVHTVPWDQLFRNPHQALLHTANRPEDECVGEGLACHQLCARGHCWGPGP
TQCVNCSQFLRGQECVEECRVLQGLPREYVNARHCLPCHPECQPQNGSVTCFGPEADQCVACAHYKDPPFCVARC
PSGVKPDLSYMPIWKFPDEEGACQPCPINCTHSCVDLDDKGCPAEQRASPLTSIISAVVGILLVVVLGVVFGILI
KRRQQKIRKYTMRRLLQETELVEPLTPSGAMPNQAQMRILKETELRKVKVLGSGAFGTVYKGIWIPDGENVKIPV
AIKVLRENTSPKANKEILDEAYVMAGVGSPYVSRLLGICLTSTVQLVTQLMPYGCLLDHVRENRGRLGSQDLLNW
CMQIAKGMSYLEDVRLVHRDLAARNVLVKSPNHVKITDFGLARLLDIDETEYHADGGKVPIKWMALESILRRRFT
HQSDVWSYGVTVWELMTFGAKPYDGIPAREIPDLLEKGERLPQPPICTIDVYMIMVKCWMIDSECRPRFRELVSE
FSRMARDPQRFVVIQNEDLGPASPLDSTFYRSLLEDDDMGDLVDAEEYLVPQQGFFCPDPAPGAGGMVHHRHRSS
STRSGGGDLTLGLEPSEEEAPRSPLAPSEGAGSDVFDGDLGMGAAKGLQSLPTHDPSPLQRYSEDPTVPLPSETD
GYVAPLTCSPQPEYVNQPDVRPQPPSPREGPLPAARPAGATLERPKTLSPGKNGVVKDVFAFGGAVENPEYLTPQ
GGAAPQPHPPPAFSPAFDNLYYWDQDPPERGAPPSTFKGTPTAENPEYLGLDVPV
Amino acid sequence of a Fab heavy chain domain of trastuzumab
modified at position L₁₁₅.
SEQ ID NO: 27
EVQLVESGGG LVQPGGSLRL SCAASGFNIK DTYIHWVRQA PGKGLEWVAR IYPTNGYTRY
ADSVKGRFTI SADTSKNTAY LQMNSLRAED TAVYYCSRWG GDGFYAMDYW GQGTXVTVSS
ASTKGPSVFP LAPSSKSTSG GTAALGCLVK DYFPEPVTVS WNSGALTSGV HTFPAVLQSS
GLYSLSSVVT VPSSSLGTQT YICNVNHKPS NTKVDKKVEP
Amino acid sequence of a light chain domain of trastuzumab
modified at position Q₁₆₀.
SEQ ID NO: 28
DIQMTQSPSS LSASVGDRVT ITCRASQDVN TAVAWYQQKP GKAPKLLIYS ASFLYSGVPS
RFSGSRSGTD FTLTISSLQP EDFATYYCQQ HYTTPPTFGQ GTKVEIKRTV AAPSVFIFPP
SDEQLKSGTA SVVCLLNNFY PREAKVQWKV DNALQSGNSX ESVTEQDSKD STYSLSSTLT
LSKADYEKHK VYACEVTHQG LSSPVTKSFN RGEC
Amino acid sequence of a light chain domain of trastuzumab
modified at position E₁₉₅.
SEQ ID NO: 29
DIQMTQSPSS LSASVGDRVT ITCRASQDVN TAVAWYQQKP GKAPKLLIYS ASFLYSGVPS
RFSGSRSGTD FTLTISSLQP EDFATYYCQQ HYTTPPTFGQ GTKVEIKRTV AAPSVFIFPP
SDEQLKSGTA SVVCLLNNFY PREAKVQWKV DNALQSGNSQ ESVTEQDSKD STYSLSSTLT
LSKADYEKHK VYACXVTHQG LSSPVTKSFN RGEC

### EQUIVALENTS

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

1. A VEGF-A-binding protein comprising:
a heavy chain domain having an amino acid sequence of SEQ ID NO: 2; and
a light chain domain having an amino acid sequence of SEQ ID NO: 5, SEQ ID NO: 6, SEQ ID NO: 7, SEQ NO NO: 12, SEQ ID NO: 13, SEQ ID NO: 14 or SEQ ID NO: 15.

2-8. (canceled)

9. A VEGF-A-binding protein comprising:
a heavy chain domain having an amino acid sequence of SEQ ID NO: 4; and
a light chain domain having an amino acid sequence of SEQ ID NO: 1, SEQ ID NO: 5, SEQ ID NO: 6, SEQ ID NO:7, SEQ NO NO: 12, SEQ ID NO: 13, SEQ ID NO: 14 or SEQ ID NO: 15.

10-17. (canceled)

18. A VEGF-A-binding protein comprising a heavy chain domain having an amino acid sequence of SEQ ID NO: 4, SEQ ID NO: 5, SEQ ID NO: 6, SEQ ID NO: 7, SEQ ID NO: 12, SEQ ID NO: 13, SEQ ID NO: 14, SEQ ID NO: 15.

19-25. (canceled)

26. The VEGF-A-binding protein of claim 1, wherein the protein comprises an Fc domain having an amino acid sequence of SEQ ID NO: 3.

27. The VEGF-A-binding protein of claim 1, wherein the protein is in the form of a antibody, a Fab antibody fragment, a single-chain Fv antibody fragment, a minibody, or fusion protein.

28. The VEGF-A-binding protein of claim 27, wherein the protein is in the form of a monoclonal antibody.

29. The VEGF-A-binding protein of claim 28, wherein the monoclonal antibody is a chimeric monoclonal antibody, human monoclonal antibody or humanized monoclonal antibody.

30. (canceled)

31. (canceled)

32. The VEGF-A-binding protein of claim 1, wherein the protein is conjugated to a diagnostic agent or a therapeutic agent.

33. The VEGF-A-binding protein of claim 1, wherein the protein is hyperglycosylated.

34. A composition comprising the VEGF-A-binding protein of claim 1.

35. The composition of claim 34, wherein the protein is present at a concentration of 50 mg/ml to 250 mg/ml.

36-39. (canceled)

40. The composition of claim 1, wherein the composition further comprises at least one buffer, at least one stabilizer, and/or at least one surfactant.

41. The composition of claim 1, wherein the composition is liquid.

42. The composition of claim 41, wherein the composition is formulated for subcutaneous injection.

43. (canceled)

44. The composition of claim 1, wherein the composition further comprises histidine HCl, trehalose dehydrate, methionine and/or polysorbate.

45. (canceled)

46. A method of treating a condition in a subject in need thereof, comprising administering to the subject a therapeutically effective amount of the VEGF-A-binding protein of claim 1.

47. (canceled)

48. (canceled)

49. A nucleic acid encoding the VEGF-A-binding protein of claim 1.

50. A vector, an expression cassette, or a host cell comprising the nucleic acid of claim 49.

51-52. (canceled)

53. A method of producing an antibody, comprising culturing the host cell of claim 50.

54. A kit comprising a container and the VEGF-A-binding protein of claim 1.

55-59. (canceled)

60. A HER2-binding protein comprising:
(a) a heavy chain domain having an amino acid sequence of SEQ ID NO: 21, and a light chain domain having an amino acid sequence of SEQ ID NO: 24 or SEQ ID NO: 25;
(b) a heavy chain domain having an amino acid sequence of SEQ ID NO: 23, and a light chain domain having an amino acid sequence of SEQ ID NO: 20; or
(c) a heavy chain domain having an amino acid sequence of SEQ ID NO: 23, and a light chain domain having an amino acid sequence of SEQ ID NO: 24 or SEQ ID NO: 25.

61-66. (canceled)

67. A HER2-binding protein comprising a heavy chain domain having an amino acid sequence of SEQ ID NO: 23, SEQ ID NO: 24, or SEQ ID NO: 25.

68-103. (canceled)
